SEQUENCE LISTING (53 sequences)

SEQ ID NO: 1 - protein, 327 aa, Blautia obeum ATCC 29174
MNPLTLAQEIIDGRRITREDDLSFFLTCDLDELCEGADRIREACIGDKVDLCSIINGRSGRCPEDCKYCAQSAHHHTSCEVYNFLPEEKILEACKMNESEGVDRFSIVTAGKALTGKEFDQAIHAYETMHRECKIDLCASMGFISAEQLHRLHEAGVTSYHHNIETSRRNFPNICTTHTYDMKIETLKKVKAEGMCACSGGIIGMGETWEDRLDMAISLAELGIDSIPINALMPIPGTPLEHLPELSEPDILRTIAFFRYINPEANIRLAAGRALLTNDGETAFKAGASASITGNMLTTVACATIRSDRKMLADMGRDVTPEYWKEV

SEQ ID NO: 2 - protein, 325 aa, Clostridium sp. HMSC19B10
MEKYIIKLKNKVLREKEISYEEALNLISLDTNNKNDFDILLKSANEIREYFMGRKADLCTIMNAKSGKCSEDCKFCAQSSHYKTGVEEYSLLDYNEILNRAKEMESKGVHRFSLVTSGKGMSGKEFNNILNIYEGLRENTNLKLCASLGIIDYEQAKMLKSAGVTTYHHNVETCRDNFHNICTTHTYKDRIKTIKDAKKAGLDVCVGGIIGMNESEEQRLKMVFEIRELNVKSFPINILNPIKNTPMENYDVLEPMEILKTTAVFRFIIPNVYIRYAGGRLSLKGYDKVGFNGGVNSAIVGDYLTTVGSGIENDKKMIIEQGFEL

SEQ ID NO: 3 - protein, 813 aa, Bacteroides caccae ATCC 43185
MKKQRHIQTTRTLLSRFRYWGRKNYAAFASMGREFQIGHLHTNVVDVALRKQNAAQTIPYHTFMTLQEIKDQVLAGIDISPDQAAWLANMADSEALYAAAHEITVARASHEFDMCSIINAKSGRCPENCKWCAQSSHYKTQAEVYDLLPAKECLRQAKYNETQDVNRFSLVTSGRKPSSRQITQLCDTVRHIRRHSSIQLCASLGLLDENELHALHDAGITRYHCNLETAPSYFPKLCSTHTQEQKLATLNAARRVGMDICCGGIIGMGETMEQRIEFAFTLKALNVQSIPINLLSPIPGTPLENEKPLSEEKVLRTIALFRFINPTAFLRFAGGRSQLSSEAMSKALYIGINSAIVGDLLTTLGSKVSEDKQMILKEGYQFASSQFDREHLWHPYTSTTDPLPVYKVKRADGATITLENGRTLIEGMSSWWCAVHGYNHPALNQAAKDQLDKMSHVMFGGLTHDPAIELGKLLLPLVPPSMQKIFYADSGSVAVEVALKMAVQYWYAAGKPDKNNFVTIRSGYHGDTWNAMSVCDPVTGMHSLFGASLPVRYFVPAPSSRFDGEWNPQDIEPLRKTIEKHSEELAALILEPIVQGAGGMWFYHPQYLREAEKLCKEHGLLLIFDEIATGFGRTGKLFAWEYAGVEPDIMCIGKALTGGYMTLSAVLASNHVADTISNHAPGAFMHGPTFMGNPLACAVACASVRLLLQSGWQENVKRIETQLKEELAPARELPQVADVRILGAIGVIEMKRPVNMAFMQRRFVEEGIWVRPFGKLVYLMPPFIITPEQLSKLTTGLLKVISGERNKIYINNL

SEQ ID NO: 4 - protein, 319 aa, [Clostridium] spiroforme DSM 1552
MLKKILNKVLNEQEISNDEAISLINVDLEELKKAANQIRKHFCGNKFDLCSIINGKSGKCKEDCKFCGQSIHYHTNIQTYDLLNSDEIKKMALYNEKKGVQRFSIVTSGRCLNNQEFENILKIYQELNQSTNISLCASLGLLKYHQLKKLKDVKVIKYHNNLETSQKYFKEICTTHTYQQKIETINDASNAGLQVCSGGIVGMNETWQDRIDLAFKLNQLKIMSIPINILNPIKNTPLEKIKPLSKEEIERIFAIFRFINPKAIIKIAGGRELLNDKGLGVFKSGANGAITGDMLTTKGISIDDDIEIINELGFEIKKI

SEQ ID NO: 5 - protein, 330 aa, Porphyromonas gingivalis (strain ATCC BAA-308 / W83)
MIQTIENRLINGGELTLEEALLLASSADKEALYEAAHRITRYFMGDKFDTCSIINAKSGNCPEDCKWCAQSRHYAASIEKYGLLSAAVCAEQAAYNRRQGIGRFSLVASGRTASMNEIRQMAESYRAIKQRTDIKCCASLGLLSEEKLQILFDNGVTTYHCNMETAPSFFPSLCSTHTQDEKLATIRAARRVGMRVCSGGIIGMGETMEQRIEFAFYLHSINVYSIPINILQPIPGTPLERTLPLSEEEYLTTVALFRLINPRAFLRFSGGRAQLSPAVQRKAIYIGINAAITGDLLTTTGSKAAEDMQLVRECGFQVTNDTDWEVSYDH

SEQ ID NO: 6 - protein, 752 aa, Bacteroides cellulosilyticus DSM 14838
MTVEELKNKVLQDVPITQAEAEWLATQPDKEALYEAAHEITAARASQEFDMCSIINAKSGRCPENCKWCAQSSHYKTKADVYDLVDKDECLRHALHNESQGVARFSLVTSGRKPSGKNMEKLCEAARYMRRKSSIQLCASLGLLNEDELRALHTAGVTRYHCNLETAPSHFPTLCSTHTQEEKLQTLAAARRVGMDICCGGIIGMGETAGQRIEFAFTLRDLEVPSIPLNLLQPIPGTPLESQSRLTEEEVLTTIALFRFINPTAYLRFAGGRSQLTKEAVKKSLYIGVNSAIVGDLLTTLGSKVSEDKALIEDAGYRFSDSQFDREHLWHPYTSTTNPLPVYKVKRGDGATITLENGEVLIEGMSSWWCMVHGYNNPVLNQALREQTEKMSHVMFGGLTHDPAIELGKLLLPLVPPSMQKIFYADSGSVAVEVAMKMAVQYWYAKSQSAPGRAHLAKKSNFVTIRRGYHGDTWNAMSVCDPVTGMHSLFGSALPVRYFTSRPQSRFDGDWNAEDTLELLDIVEKHHEELAALILEPIVQGAGGMWFYHPQFLREAARICKEYNILLIFDEIATGFGRTGKMFAWEYAGVEPDIMCIGKALTGGYMTLSAVLTTNEVADSISNHAPGAFMHGPTFMGNPLACAVACASIRLLTSSEYDWQGKVSRIEEQLWRELEPALALPQVADVRVLGAIGVIEVQDPVDMAWMQKRFVEEGIWVRPFGKLVYIMPPFIIEPEQLTKLTSGLLKIIGEMG

SEQ ID NO: 7 - protein, 319 aa, Clostridium perfringens (strain ATCC 13124 / DSM 756 / JCM 1290 / NCIMB 6125 / NCTC 8237 / Type A)
MDFVLKMKDKSLKNRKLTREEGLRLFNSNLEELIKEANNIRKEIHGDGIDLCSIINGKSGRCGEDCAFCAQSKYHKTNISEYPLLDYEKIKKVAKENEDEGVHRFSIVTSGRGLYGEEFERVITYYSNLNKELKINLCASHGIINKESLIKLKKAGVKRYHHNLETSRNCYDKICKTHSYEERVKTIKNAKEAGLEVCSGGIIGLGETIIDRIDLAITLRELEIKSIPINVLSAIKGTKLQHMIPLNEEEILRTIAVFRFINPEAKIRLAGGRYLLKNFGENAFKAGANATITGNLLTTCGNKIKDDKRLIENIGMRIF

SEQ ID NO: 8 - protein, 320 aa, Clostridium thermocellum (strain ATCC 27405 / DSM 1237 / NBRC 103400 / NCIMB 10682 / NRRL B-4536 / VPI 7372)
MDFLTLMENKLDEGRMITFEEAVELAKGKIEDEKLFLLADKLCKKNMGRRVDLCSIINAKSGGCSENCKFCAQSGHYDTGVKIYPLLDVDDVLKAAKENEKEGVHRFSLVTSGKSVSDEEFEKILGIYSVLRKETNLKLCASLGSLSYERAVRLKEAGVSMYHHNIETCREYYPKICDTHTYDDRINTVKNAAKAGLEICCGGIIGMGESMEQRIKMAFEIRELGVKSVPINVLNPIKGTPFENVRSLSPDEILRTIALFRLIMPYAHIRYAGGRMCLGEHQSKGFKAGVSAMLVGNYLTTVGNKISDDLEMIQRMGLEI

SEQ ID NO: 9 - protein, 342 aa, Veillonella parvula HSIVP1
MALPIDQQPSQVGTYEKILTIANRIMNGGEITKEEAIELIHTSDDDTMILLAMADKIRQHFNDNSVDVCAIVNARSGKCPENCKFCAQSAHHNTGVQEYPFMDEESILQAARKAKEAGAIRFSIVTSGRNTNNPDEFDQIIHVLGRIKNEIGLEICCSLGLLTYEQALKLKEVGVTRYHSNIETAPSHFPDICTTHSYEDKMFTIDNAQKAGIRVCSGGILGLNETLEQRVEMAFELKRLHIDSVPLNILNPVKGTPFESNEALRPLDILRTFAVFRFILPNALIRTAGGREVNLRDLQAYALKGGLNGIMVGGYLTTGGRSPQDDLQMIQDLELTRNTAQV

SEQ ID NO: 10 - protein, 382 aa, Cyanothece sp. (strain ATCC 51142)
MIQFIRPKTASGKTIVVQLSASPPQTPPPTDTNALKNWLEILSQRIINGDRLTKTEALTLTQIDGQDNILLLCEAADRIRQACCGNVVDLCSIINVKSGNCSENCQFCSQSVHHQGENSPIYGLKSSEEILAQAKAAEVAGAKRFCLVSQGRGPKYNSPQSGEFEKILETVRQITTETNIKPCCALGEVTLEQAEALKEAGVTRYNHNLEASENYYQSIVSTHSWGDRVETVKNLKKAGIQACTGGIMGMGETWEDRVDLAISLRELEVDSVPINLLNPREGTPLGHLPKLDVFDALKAIAIFRFLLPEQILRYAGGREAVMGELQSLGLKSGINAMLIGHYLTTLGQPPENDHAMLESLGLQGGEAPIPGEYKKKTQKKAH

SEQ ID NO: 11 - protein, 330 aa, Porphyromonas gingivalis (strain ATCC 33277 / DSM 20709 / CIP 103683 / JCM 12257 / NCTC 11834 / 2561)
MIQTIENRLINGGEPTFEEALLLASSADKEALYETAHRITRHFMGDKFDTCSIINAKSGNCPEDCKWCAQSRHYATSIEKYGLLSATVCAEQAAYNRRQGIGRFSLVASGRTASMNEIRQMAESFRTIKQRTDIKCCASLGLLSEEKLQILFDNGVTTYHCNMETAPSFFPSLCSTHTQEEKLATIRAARRVGMRVCSGGIIGMGETMEQRIEFAFFLHSINVYSIPINILQPIPGTPLEKTPPLSEEEYLTTVALFRLINPRAFLRFSGGRAQLSPAVQRKAIYIGINAAITGDLLTTTGSKAAEDMQLARECGFQVTNDTDWEVSYDH

SEQ ID NO: 12 - protein, 321 aa, Akkermansia muciniphila (strain ATCC BAA-835 / Muc)
MSLTSSLLSRVLGGGSCSREELIALSREPLEELCQAANAIREHFCGNVFDLCTIINGRSGKCSENCKYCAQSAHYSTAVEEYPLLSDEALLAGARYNDARGILRYSIVTSGKRLTDEDVDRLCASYRHIAEHCGISLCASHGLISKKHCEQLKAAGVSRYHNNLETSRRNFPNVCTTHTYDDKLQTIKWALEAGLEVCSGGIMGLGETMEDRIDMYMDIAALGIKSMPVNFLTPIPGTPYADMTPLGEEEQLRIVALVRFIMPDGFVRIAAGRNTMKDHGRKIFMSGANAAISGDMLTTAGVTIREDLAMLAELGYEVRMK

SEQ ID NO: 13 - protein, 346 aa, E. coli K12 P12996-1
MAHRPRWTLSQVTELFEKPLLDLLFEAQQVHRQHFDPRQVQVSTLLSIKTGACPEDCKYCPQSSRYKTGLEAERLMEVEQVLESARKAKAAGSTRFCMGAAWKNPHERDMPYLEQMVQGVKAMGLEACMTLGTLSESQAQRLANAGLDYYNHNLDTSPEFYGNIITTRTYQERLDTLEKVRDAGIKVCSGGIVGLGETVKDRAGLLLQLANLPTPPESVPINMLVKVKGTPLADNDDVDAFDFIRTIAVARIMMPTSYVRLSAGREQMNEQTQAMCFMAGANSIFYGCKLLTTPNPEEDKDLQLFRKLGLNPQQTAVLAGDNEQQQRLEQALMTPDTDEYYNAAAL

SEQ ID NO: 14 - protein, 346 aa, Escherichia coli; identical to SEQ ID NO: 13

SEQ ID NO: 15 - protein, 346 aa, Candidatus Chloracidobacterium thermophilum B
MSQPLVRFDWTRDELRALHDLPLLELIHRAATVHRTCHDPQEVQVCRLISIKTGGCPEDCGYCSQSAHYETGIAAQPLLDKATVVAIAERAKAHGVSRVCLGAAWRNVRDDAQFEAVLDIVRSVNALGIEVCCTLGMLTEAQARRLEEAGLYAYNHNLDTSREYYGRVVTTRTYDDRLETLANVRKTGVTLCTGGILGLGESTDDRIGLLHTLATMNPHPESVPINLLTRVPGTPMENEAEVSVWETLRVIATARIAMPRSVIRLSAGRTQLSEEAQALCFLAGANSIFSSDARMMLTRVSPTNDYDEDAQLLNKLGLHPRVPFKDAPNAKTAGCASAATATLQEK

SEQ ID NO: 16 - protein, 387 aa, Streptomyces lydicus
MPYVRINAMDLLNTLVDKGLRRELPTREEALAVLATSDDELLDVVAAAGKVRRQWFGRRVKLNYLVNLKSGLCPEDCSYCSQRLGSKAEILKYTWLKPDDASKAAAAGVAGGAKRVCLVASGRGPTDKDVDRVSETISAIKEQNEGVEVCACLGLLSDGQADRLRAAGADAYNHNLNTSEATYGDICTTHDFSDRVSTVQQAQAAGMSACSGLIAGMGESDADLVDVVFALRELDPDSVPVNFLIPFEGTPLAKEWNLTPQRALRILAMVRFVCPDVEVRLAGGREVHLRSLQPLALHLVNSIFLGDYLTSEGQAGKEDLAMIADAGFEVEGTDTTTLPEHRTDAAVQPAPEPAADAAVPAPPSEETRRDLVSVRRRGAGTELPPNA

SEQ ID NO: 17 - protein, 324 aa, Paracoccus denitrificans
MIRTDWTMAEAWAIHALPFADLMHRAQTLHRAHFDPNAIETASLLSIKTGGCPEDCGYCSQSAHHDTGVKATKLMGTEEVLAAARRAKASGAQRFCMGAAWRSPKDRDMDKLCDMVRGVAELGLETCMTLGMLSPEQVARLKAAGLDFYNHNIDTSPEYYAQIASTRTMENRLDTVEQVRKGGIKVCCGGILGMGEAEEDRIAMLVTLATLPAHPDSVPVNLWNEIEGVPVQARAQAVDPFALVRIVALARILMPASVVRLSAGRTGMSDELQALCFLAGANSIFVGDQLLTTGNPAAWKDQDLLSRLGMHIAPAQARPRVAAE

SEQ ID NO: 18 - protein, 320 aa, Paracoccus denitrificans
MPQQSRSAAEIYHQPLMDLLFQAQTVHRAHFDPNVVQCSKLLSIKTGGCPEDCAYCSQSARNGSELSASKLMEVQRVLAEARRAKEAGATRYCMGAAWRSPKERDMPAVLAMIRGVKAMGMETCMTLGMLDADQALRLKDAGLDYYNHNIDTSERYYSEIITTRTFQDRIETLERVQAAGINVCAGGIVGMGETAEDRISMLETLAGLEVPPQSVPINMLMPMAGTPLADVPRLDAIEMVRTIATARILMPASYVRLSAGRSEMSDEMQAMCFFAGANSIFVGDTLLTAGNPDEDKDALLFAKLGLRAEVPEASQEGCAA

SEQ ID NO: 19 - protein, 328 aa, Agrobacterium vitis
MSEAAGEIRNDWSVEEIVTLHNLPLLELIGHANAVHGRHHNPNVVQKASLLSIKTGGCPEDCAYCPQSAHHREVKLTKDRLMQPETVLALAKRAKDAGAERFCMGAAWRQVRDGKEFDAVLTMVRGVRDLGMEACVTLGMLEKHQAEKLAEAGLTAYNHNLDTSPEFYGEIITTRSYADRLETLSIVRSFGIDLCCGGIIGMGETIRDRASMLQVLASMRPHPESVPINALVPVEGTPLAAMPRIDPLELVRMVATARIVMPKSTVRLSAGRSTLNREAQILCLVSGANSVFYGDTLLTTPNAGIGEDEALFAAIGALPHEAAPLAAE

SEQ ID NO: 20 - protein, 318 aa, Ruegeria pomeroyi
MAEAIRSDWSVDEVEALLRLPLLDLVGRANGVHRAHHAPDDIQKASLLSIKTGGCPEDCAYCPQSAHHREVELTREKLMNPDHVVSLARRAQRAGAERFCMGAAWRQVRDGAEFDNVLAMVRGVRALGMEACVTLGMLRPHQAQRLAEAGLTAYNHNLDTSPEFYGQIIGTRTYQDRLDTLAYCRDAGIELCCGGIIGMGESLRDRAAMLQVLANFAPHPESVPINALIPIEGTPLAHRERVGIFDLVRMVATARIIMPLTRVRLSAGRSDFSAAEQALCFLAGANSVFYGDVLLTAPNAGTGADAELFAALGALETA

SEQ ID NO: 21 - protein, 339 aa, Agrobacterium fabrum
MDQLATQIDGKPASIPAVETSSSLEEAKIIYNLPFNDLLFRAQQVHRCHFDANAIQMSRLLSIKTGGCPEDCSYCSQSARNPTGLKASKLMEVERVLAEARKAKEGGATRYCMGAAWRNPKERDMEAVVAMVEGVKALDMETCMTLGMLTPEQSERLADAGLDYYNHNVDTSERFYSEIITTRTFEDRLETLANVRDAGIKVCAGGILGMGETVEDRISMLVTLANLPVPPESVPINMLIPIPGSKLANADPVDPIDFVRTIALARILMPRSHVRLSAGRTEMSDETQALCFLAGANSIFIGETLLTADNPGEDHDTALFRRLGLKPMELQSSEAGGCR

SEQ ID NO: 22 - protein, 317 aa, Wolbachia endosymbiont of Cimex lectularius
MTEEWTFAKADQIFNFPFPELIYIAQTEHRKQFNPSEVQISTLLSIKTGSCPENCSYCPQSAHYNTGLQKKPLLEIAEVIEAAKCAKEAGSTRFCMGAAWRGPRDQDLKVVCEMIREVKKLGLETCVTLGLLKDHQANMLKEAGLDFYNHNIDTSEEYYNKVITTRTFQDRLETLECVRASGIKVCCGGILGMGETNEDRIKMLVLLANLNDPPESVPINTLIKIPGTPLENVADVDPFDFVRTIAIARIIMPKSYIRLSAGREKMSDELQALCFLAGANSIFYGEKLLTAQNPIPEQDNHLFQRLGLQKLALLREN

SEQ ID NO: 23 - protein, 341 aa, Sphingomonas paucimobilis
MTTTPALSSEATPRTDWTRAEIAALFDLPFTELLFRAAEVHRAHHAADQVQLSTLLSIKTGGCPEDCGYCSQSTHADTGLKATKLMDPRAVLQAAAQAKDHGSTRFCMGAAWRNPKDRDMPAIVEMVKGVRAMGMETCMTLGMLTDAQAQTLAEAGLDYYNHNIDTSPERYGDVITTRSFGERLETLEHVRDAGINVCCGGIVGMGETRGDRVGFIHALATLPVHPGSVPVNALVPVKGTVLGDMLADTPLAKIDDIEFVRTVAVARITMPHSMVRLSAGRESMSDATQALCFLAGANSIFTGDKLLTAGNAGDDKDAALFARLGLTPMAAECKVELEAAE

SEQ ID NO: 24 - protein, 316 aa, Acidithiobacillus ferrivorans
MNTTAPPQTLDAILEIYASPFNDLIFEAQKVHRLHFDPNAIQCSTLLSIKTGGCPEDCGYCSQSAHHQTALKAEALMDLEQVRAAAQEAKANGAQRLCMGAAWRSPHDKDIEKVAAMIGVVKEYGLESCVTLGMLKPGQAERLQNAGLDYYNHNLDTSPEFYGEVIHTRSYQDRLDTLETVRSAGIKICSGGILGMGESRRDRARMLQILAHLPQAPESIPINALVPVPGTPLEAAEPIDGFEFVRTIAVARILFPKAYVRLSAGRGAMSDELQALAFLAGANSIFLGDRLLTTDNASMGHDQSLFSRLGLHRSEA

SEQ ID NO: 25 - protein, 331 aa, Gallionella capsiferriformans
MNTQTIAFHHPVKRTATPERWSVEAVESLFALPFADLLYRAQQVHREHFDPNQVQLSTLLSIKTGGCSEDCGYCPQSAFHSTGVEDRKMLALDAVIEAAKAAQAAGADRFCMGAAWREPSEADMLSVVDMVQAVRGLGMETCATLGMLNDAQTEQLRAAGLDYYNHNLDTSPEFYGDIISTRDYQDRLDTLERVRRAGMHVCSGGIVGMGESLTERAGLVAQLANLNPYPESVPINNLVKVEGTPLADAAELDPLDFVRTIAVARITMPTARVRLSAGRQAMSDAIQALCFLAGANSIFYGEQLLTTGNPEVERDRALMDKLGMYPFADKH

SEQ ID NO: 26 - protein, 342 aa, Ralstonia eutropha
MNQAAQTVATISAEALRQTARNTHALPEDARWRVDDVAALFALPFNDLLFRAQQVHRENFDANTVQLSTLLSIKTGGCEEDCGYCPQSAHHDAGVKAEKLMELDEVLEAARAAKANGATRFCMGAAWRSPKDRHLEPVMDMVREVKAMGLETCVTLGMLKAEQAQQLKDAGLDYYNHNLDTSPEFYGKIITTRTYQDRLDTIGHVRDAGINVCCGGIVGMGESREARAGLIAQLANMDPYPESVPINNLVQVEGTPLAGTEALDPFEFVRTIAVARITMPGAMVRLSAGREAMDEALQALCFMAGANSIFYGEKLLTTGNPQADRDRALLARLDIRAEGYAG

SEQ ID NO: 27 - protein, 335 aa, Bordetella parapertussis
MHTAYIPVPTPVRPPSAERWPLAAVAELFELPFLDLLHRAQQVHRQHFDANTVQLSSLLSIKTGGCPEDCAYCPQSAHYDTGVDADKLMPLDEVVRAARAAQANGAQRFCMGAAWRSPKPHHLEAVAEMIGAVKALGMETCVTLGMLRDGQAEQLKAAGLDYYNHNLDTAPEFYGKIISTRTYQDRLDTLQQVREAGINVCCGGIVGMGESRRDRAGLVAQLANMEPYPESVPINNLVQVEGTPLAGAETLDPFEFIRTIAVARITMPLAKVRLSAGRETMSDSEQALCFMAGANSIFYGDVLLTTGNPQVEADRRLLQRLGMRAEGLPCAAGQA

SEQ ID NO: 28 - protein, 347 aa, Pusillimonas sp. T7-7
MAAMKPAIPSHTPTPDHAPQAWGIAQILRLYELPFLDLLHQAQAVHRAHHQPNTVQLSSLLSIKTGACPEDCAYCPQSARHDTGGKQEALMPVAEVLEAARKAKANGAQRFCMGAAWRSPTARQLDSVVEMVGAVKALGLETCVTLGMLKEGQAERLRDAGLDYYNHNLDTSPEFYGNIITTRSYQDRLDTLERVRNAGVHVCCGGIVGLGESRKERAGLVAQLANLSPYPESVPVNNLVKVAGTPLDATPDIDPFEFVRTIAVARITMPRAVVRLSAGREAMSDAIQALCFMAGANSIFYGEQLLTTANPQLSQDQQLFQRLGLTATPADPARPAHLEHHHEATLA

SEQ ID NO: 29 - protein, 318 aa, Cenarchaeum symbiosum A
MGIAECRDKVLGGGELTKDEARGLMEADVTELAAAADEITRRFNGDGVDVEQLNNIKRDGCSEDCTFCGQSAFYDADKEPHPLPEPEEVVRAALKAKKEEASSYCLVAAWREPTPEGFEKVCTIIQEINTHVGISVECSLGFLTRERAARLKGLGVKRYNHNLETARSKFPEICSTHTYEDRLDTLEIAREAGLELCTGGIIGMGESRGQRIELAMELARIRPEEATVNILVPVQGTPMELQAPLPPGEAERFFALVRFLLPRSVVKISGGREKALDDDGRAILRGGANGIITSGYLTMGGNDSSADMEMIREAGLEA

SEQ ID NO: 30 - protein, 332 aa, Alicyclobacillus acidocaldarius subsp. acidocaldarius DSM 446
MMKIDYQTNWIDLARRVLDGRGVTREEALDILRSSDDELLDLLAAAFLIRRRYFGKKVKLNMIINAKSKMCPEDCAYCSQSAISKAPVSKYPLVSKEEIIAGAREAERRKAGTYCIVISGRRPSDREIERIAEAVEEIRATTTLKICCCLGLLTPAQADRLARAGVHRYNHNLNTSRDRYGDICTTHTYDDRVRTLEHVKEAGISPCSGVIFGMGESDEEAVDMAFALKEMDADSIPCNFLNPIPGTPLEGMETLNPRRCLKLLCMMRFVNPSKEIRIAGGRERNLRSLQVLGLYPANSIFVGDYLTTPGQAPTEDWAMIEDLGFEIEECAL

SEQ ID NO: 31 - protein, 331 aa, Geobacillus thermoglucosidasius C56-YS93
MINWLALADRVIAGHELTDEEALAILDCPDEELLLLMQGAYNIRRTYYGNKVKLNMIINAKSGLCPENCGYCAQSAVSTAPVKTYKMVDKETLIRGAEEAYRMRIGTYCIVASGRGPSEKEIDTVVSAVKEIKERFGLKICACLGILKPEQAARLKEAGVDRYNHNINTSKEHHPNITTSHTYDDRVRTVETVKQAGISPCSGVIIGMRETKQDVINMARSLRILDADSIPVNFLHAIDGTPLAGTNELDPRYCLKVLALFRYMNPTKEIRIAGGREVNLRSLQPLGLYAANSIFVGDYLTTAGQEKSEDYRMLEDLGFEIDFAEEQQVVC

SEQ ID NO: 32 - protein, 335 aa, Bacillus subtilis subsp. subtilis str. 168
MNQWMELADRVLAGAEVTDEEALSILHCPDEDILLLMHGAFHIRKHFYGKKVKLNMIMNAKSGLCPENCGYCSQSAISKAPIESYRMVNKETLLEGAKRAHDLNIGTYCIVASGRGPSNREVDQVVDAVQEIKETYGLKICACLGLLKPEQAKRLKDAGVDRYNHNLNTSQRNHSNITTSHTYDDRVNTVEIAKESGLSPCSGAIIGMKETKQDVIDIAKSLKALDADSIPVNFLHAIDGTPLEGVNELNPLYCLKVLALFRFINPSKEIRISGGREVNLRTLQPLGLYAANSIFVGDYLTTAGQEETEDHKMLSDLGFEVESVEEMKASLSAKS

SEQ ID NO: 33 - protein, 331 aa, Lysinibacillus sphaericus
MNFLQVAQEVIDGKIISNEEALAILNSKDDELLQLMDGAFAIRRHYYGKKVKLNMIMNAKSGYCPEDCGYCSQSSKSTAPIEKYPFITKEEILAGAKRAFDNKIGTYCIVASGRGPTRKDVNVVSEAVTEIKEKYGLKVCACLGLLKEEQAQQLKEAGVDRYNHNLNTSERHHSFITTSHTYEDRVNTVEIVKKHGISPCSGAIIGMKETREDVVNIARALHQLDADSIPVNFLNAIDGTKLEGTRDLNPRYCLKVLALFRYINPTKEIRISGGREINLGSLQPLGLYAANSIFVGDYLTTAGQEANSDYRMLEDLGFEIELTQKQEAAFC

SEQ ID NO: 34 - protein, 333 aa, Methylococcus capsulatus str. Bath
MHAEVAVMTNQERAEEPVLRHDWTQGEAEALFALPFNELLFQAQTIHRRHFDPNEVQVSSLLSIKTGACSEDCAYCPQSAHYETGVKRESLMSLEDVLEAAQRAREEGATRFCMGAAWRSPRDGDLEAIAAMVEGVKALGMETCVTAGMLSDEQARRLKEAGLDYYNHNLDTSESYYGEIITTRTYQDRLDTLQRVRDAGMHVCCGGIVGMGESAADRAGLLIGLANLPRHPESVPINLLVRVEGTPLADTAALDPFDFVRTVAVARIMMPASRVRLSAGRSDMSDEMQALCFLAGANSIFYGDRLLTTENPQAQRDRRLFARLGLRMAGLGC

SEQ ID NO: 35 - protein, 346 aa, Leclercia adecarboxylata
MAHQTRWTLSQVTALFEKPLLELLFEAQQIHRQHFDPQQIQVSTLLSIKTGACPEDCKYCPQSARYKTGLESERLMEVEQVLESARQAKNAGSTRFCMGAAWKNPHERDMPYLEQMVQGVKAMGLEACMTLGTLDDTQAQRLASAGLDYYNHNLDTSPEFYGNIITTRTYQERLDTLDKVRDAGIKVCSGGIVGLGETVTDRAGLLLQLANLPTPPESVPINMLVKVKGTPLADNDDVDAFDFIRTIAVARVMMPTSFVRLSAGREQMNEQTQAMCFMAGANSIFYGCKLLTTPNPEEDKDVQLFRKLGLNPQQTAVLTGDNEQQHQLEQQLINADTDQFYNAATV

SEQ ID NO: 36 - protein, 370 aa, Chromohalobacter salexigens DSM 3043
MTAQSRDPAWTDASPTFQPTMRHDWSLEEIEALFALPFNDLLFRAQQVHRAHFDPNAVQVSTLLSIKTGACPEDCKYCPQSGHYNTGLGKEKLLEIEKVVEQARAAKAAGASRFCMGAAWRSPREKDLRVVTEMVGRVKALGLETCMTLGMVDVDQARRLAEAGLDYYNHNLDTSPDYYGEIITTRTYADRLETLANVREAGMKVCSGGILGMGEAPRDRAALLQQLVRLDPHPESVPINMLVKVPGTPMENVEDMDPLTFIRAIAVARILMPKSHVRLSAGREQMDESTQALAFLAGANSIFYGDTLLTTGNPQVERDRALFDKLGLHPEPSDPHADDAHRDDEQAEIALAHAIQRQRDDALFYDATRG

SEQ ID NO: 37 - protein, 352 aa, Pseudomonas caeni
MTTSPHADTRHDWTLAEVTALLQQPFNDLIFQAQSVHRQHFNANRVQVSTLLSIKTGACPEDCKYCPQSGHYNTGLDKEKLMEVQKVLDEAKRAKEIGSTRFCMGAAWKHPSAKDLPYVLEMVKGVKAMGMETCMTLGKLDEAQTKALADAGLDYYNHNLDTSPEFYGNIITTRTYAERLQTLSYVRDAGMKICSGGILGMGESIADRAGLLIQLANLPEHPESVPINMLVKVEGTPLENAEDVDPFDFIRMLAVARIMMPKSHVRLSAGREQMNEQMQSLAFLAGANSIFYGEKLLTTANPQADKDMQLFARLGIKPEAREEYADEVHQAAIEHAIIEQRDASLFYDAATH

SEQ ID NO: 38 - protein, 352 aa, Pseudomonas monteilii
MSASTIATTRHDWTLAEVRALFQQPFNDLLFQAQSVHRAHFDANRVQVSTLLSIKTGACPEDCKYCPQSGHYNTGLEKQKLMEVQKVLEEAARAKAIGSTRFCMGAAWKHPSAKDMPYVLQMVQGVKAMGLETCMTLGRLDREQTAALAEAGLDYYNHNLDTSPEFYGSIITTRTYAERLQTLAYVRDAGMKICSGGILGMGESLDDRANLLIQLANLPEHPESVPINMLVKVAGTPLANEEDVDPFDFIRMLAVARILMPQSHVRLSAGREQMNEQMQALAFLAGANSIFYGEKLLTTGNPQADRDMQLFARLGIQPEAGEGHADEVHQAAIEQAVIEQRNGELFYDAVSA

SEQ ID NO: 39 - protein, 352 aa, Pseudomonas massiliensis CB1
MSASLNSPLRHDWTLSEVKALFTQPFNDLLFHAMSVHRAHFDPNQVQVSTLLSIKTGACPEDCKYCPQSGHYNTGLEKEKLLEVQKVIEEAARAKAIGSTRFCMGAAWKHPSAKDMPYVLEMVRGVKALGLETCMTLGRLDRDQTVALAEAGLDYYNHNLDTSPEFYGNIITTRTYGERLQTLAYVRDAGMKICSGGILGMGESLDDRAGLLIQLANLPEHPESVPINMLVKVAGTPLENAEDVDPFDFIRMLAVARILMPRSHVRLSAGREQMNEQMQALAFMAGANSIFYGEKLLTTANPQADKDMRLFARLGIRPEAREEHDDEVHQAAIEQALVEQRSGELFYDAAAV

SEQ ID NO: 40 - protein, 352 aa, Pseudomonas putida F1
MSASTTATTRHDWSLAEVKALFQQPFNDLLFQAQTVHRAHFNPNRVQVSTLLSIKTGACPEDCKYCPQSGHYNTGLEKQKLMEVQKVLEEAARAKAIGSTRFCMGAAWKHPSAKDMPYVLEMVKGVKAMGLETCMTLGKLDQEQTKALAHAGLDYYNHNLDTSPEFYGSIITTRTYSERLQTLAYVRDAGMKICSGGILGMGESLDDRAGLLIQLANLPEHPESVPINMLVKVAGTPLAEEEDVDPFDFIRMLAVARILMPKSHVRLSAGREQMNEQMQALAFMAGANSIFYGEKLLTTANPQADKDMQLFARLGIKPEAREEHADEVHQAAIEQALVEQRSSEMFYDAATA

SEQ ID NO: 41 - protein, 352 aa, Pseudomonas thermotolerans
MNASVAAAIRHDWTLAEVKALFALPFNDLLYQAQTVHRQYFDANRVQVSTLLSIKTGACPEDCKYCPQSGHYNTGLEKQKLMEVQKVLQAAAEAKAMGSTRFCMGAAWKHPSAKDFPYVLEMVKGVKALGLETCMTLGRLSREQTQALAEAGLDYYNHNLDTSPEFYGRIITTRTYAERLQTLAYVREAGMKICSGGILGMGESLDDRAGLLIQLANLPEHPESVPINMLVKVQGTPLADAEDVDPFDFIRTLAVARIMMPKSHVRLSAGREQMNEQMQALAFLAGANSIFYGEKLLTTGNPQAEKDLQLFRRLGIQPEEREEHADEVHQAAIEQALAEQRDSQLFYDAASA

SEQ ID NO: 42 - protein, 352 aa, pseudomonad ancestor
MSASTNSPLRHDWTLSEVKALFTQPFNDLLFHAMTVHRAHFDPNQVQVSTLLSIKTGACPEDCKYCPQSGHYNTGLEKEKLMEVQKVIEEAARAKAIGSTRFCMGAAWKHPSAKDMPYVLEMVRGVKAMGLETCMTLGRLDQDQTVALAEAGLDYYNHNLDTSPEFYGNIITTRTYGERLQTLAYVRDAGMKICSGGILGMGESLDDRAGLLIQLANLPEHPESVPINMLVKVAGTPLENAEDVDPFDFIRMLAVARILMPRSHVRLSAGREQMNEQMQALAFMAGANSIFYGEKLLTTANPQADKDMQLFARLGIRPEAREEHDDEVHQAAIEQALVEQRSSEMFYDAAAV

SEQ ID NO: 43 - protein, 352 aa, Pseudomonas aeruginosa PAO1
MSATASVATRHDWSLAEVRALFEQPFNDLLFQAQTVHRAHFDPNRVQVSTLLSIKTGACPEDCKYCPQSGHYNTGLDKEKLMEVQKVLEAAAEAKAIGSTRFCMGAAWKHPSAKDMPYVLEMVKGVKKLGLETCMTLGRLTQEQTQALADAGLDYYNHNLDTSPEFYGNIITTRTYSERLQTLAYVREAGMKICSGGILGMGESVDDRAGLLIQLANLPEHPESVPINMLVKVKGTPLAEEKDVDPFDFIRTLAVARIMMPKSHVRLSAGREQMNEQMQALAFMAGANSIFYGEKLLTTKNPQAEKDMQLFARLGIKPEEREEHADEVHQAAIEQALVEQRESKLFYNAASA

SEQ ID NO: 44 - protein, 352 aa, Pseudomonas balearica
MSATASIATRHDWSLAEVKALFEQPFNDLLFQAQTVHRAHFDPNRVQVSTLLSIKTGACPEDCKYCPQSGHYNTGLDKEKLMEVQKVLEAAAEAKAIGSTRFCMGAAWKHPSAKDMPYVLEMVKGVKKLGLETCMTLGRLDQEQTQALAEAGLDYYNHNLDTSPEFYGNIITTRTYSERLQTLAYVREAGMKICSGGILGMGESVDDRAGLLIQLANLPEHPESVPINMLVKVKGTPLAEEKDVDPFDFIRTLAVARIMMPKSHVRLSAGREQMNEQMQALAFMAGANSIFYGEKLLTTANPQAEKDMQLFARLGIKPEEREEHADEVHQAAIEQALVEQRDSQLFYDAASA

SEQ ID NO: 45 - protein, 351 aa, Pseudomonas fluorescens SBW25
MSASTTATLRHDWSLAEVKALFVQPFNDLLFQAQTVHRAHFDANRVQVSTLLSIKTGACPEDCKYCPQSGHYNTGLEKEKLMEVQKVLEEAARAKAIGSTRFCMGAAWKHPSAKDMPYVLQMVKGVKAMGLETCMTLGRLDQDQTEALAQAGLDYYNHNLDTSPEFYGSIITTRTYGERLQTLAYVRDSGMKICSGGILGMGESLDDRANLLIQLANLPEHPESVPINMLVKVAGTPLENAEDIDPFDFIRMLAVARILMPRSHVRLSAGREAMNEQMQALAFFAGANSIFYGDKLLTTANPQADKDMQLFSRLGILPEAREEHADEVHQAAIEQALVEQKSSEQFYNAAV

SEQ ID NO: 46 - DNA, 47 bp, Escherichia coli
  promoter (1)..(47): apFAB46 promoter
aaaaagagtattgacttcgcatctttttgtacctataatagattcat

SEQ ID NO: 47 - DNA, 37 bp, Escherichia coli
  promoter (1)..(37): apFAB70 promoter
ttgacatcgcatctttttgtacctataatgtgtggat

SEQ ID NO: 48 - DNA, 37 bp, Escherichia coli
  promoter (1)..(37): apFAB71 promoter
ttgacatcgcatctttttgtacctataatagattcat

SEQ ID NO: 49 - DNA, 29 bp, Escherichia coli
  promoter (1)..(29): pBAD Ara promoter
ctgacgctttttatcgcaactctctactg

SEQ ID NO: 50 - DNA, 74 bp, Escherichia coli
  promoter (1)..(74): lac Promoter with lacO operator site
agctgtttcctgtgtgaaattgttatccgctcacaattccacacaacatacgagccggaagcataaagtgtaaa

SEQ ID NO: 51 - DNA, 91 bp, Escherichia coli
  terminator (1)..(91): apFAB378 terminator
gagttggtagctcttgatccggcaaacaaaccaccgttggtagcggtggtttttttgtttgcaagcagcagattacgcgcagaaaaaaagg

SEQ ID NO: 52 - DNA, 91 bp, Escherichia coli
  terminator (1)..(91): apFAB377 terminator
atgaccatctacattactgagctaataacaggcctgctggtaatcgcaggcctttttatttgggggagagggaagtcatgaaaaaactaac

SEQ ID NO: 53 - DNA, 90 bp, Escherichia coli
  terminator (1)..(90): apFAB381 terminator
accctcaagagaaaatgtaaccaactcactggctcaccttcacgggtgggcctttcttcgttccgggcattaaccctcactaacaggaga

(19) United States
(12) Patent Application Publication
(10) Pub. No.: US 2022/0348974 A1
(43) Pub. Date: Nov. 3, 2022

(54) BIOTIN SYNTHASES FOR EFFICIENT PRODUCTION OF BIOTIN

(71) Applicant: ALBERT EINSTEIN COLLEGE OF MEDICINE, Bronx, NY (US)

(72) Inventors: Steven C. ALMO, Pelham, NY (US); Tyler GROVE, Bronx, NY (US)

(21) Appl. No.: 17/641,319

(22) PCT Filed: Sep. 4, 2020
(86) PCT No.: PCT/US2020/049339

§ 371 Date: Mar. 8, 2022

Related U.S. Application Data
(60) Provisional application No. 62/897,486, filed on Sep. 9, 2019.

(51) Int. Cl. C12P 17/18; C12N 9/10

(52) U.S. Cl. CPC C12P 17/186; C12N 9/13; C12Y 208/01006

(57) ABSTRACT

A recombinant microorganism includes a transgene encoding a polypeptide of a Type II biotin synthase, wherein a holo-protein of the Type II biotin synthase comprises per polypeptide chain a first [4Fe—4S] cluster (radical SAM (RS) cluster) coordinated to a CxxxCxxC motif in the polypeptide chain and a second [4Fe—4S] cluster. The Type II biotin synthase contains a serine to cysteine swap in its holo-protein amino acid sequence, that is the amino acid at the position corresponding to Ser-43 in the E. coli K12 Type I biotin synthase holo-protein is a Cysteine and the amino acid corresponding to Cys-97 in the E. coli K12 Type I biotin synthase holo-protein is a Serine. A method for producing biotin includes cultivating the recombinant microorganism in a growth medium to produce a culture; and recovering biotin from the culture.

### CROSS-REFERENCE TO RELATED APPLICATIONS

This application claims priority to U.S. Provisional Application No. 62/897,486, filed Sep. 9 2019, incorporated by reference in its entirety herein.

### STATEMENT ON GOVERNMENT SUPPORT

This invention was made in part with support from National Institutes of Health grant number P01 GM118303-01, National Institutes of Health grant number R21 AI133329, National Institutes of Health grant number U54 GM093342, and National Institutes of Health grant number U54 GM094662. The U.S. government has certain rights in the invention.

### SEQUENCE LISTING

A Sequence Listing, incorporated herein by reference, is submitted in electronic form as an ASCII text file of size 144 KB, created Sep. 2, 2020, and named “8LX7664.TXT”.

### BACKGROUND

This disclosure relates to Type II biotin synthases, and methods of producing biotin with such an enzyme.

Biotin, also known as vitamin B7, is an essential dietary vitamin for humans because humans are unable to produce biotin, which is a cofactor used in carboxylation, decarboxylation, and transcarboxylation reactions in many organisms, including humans. However, the human gut microbiome has been shown to contain Escherichia coli that contain biotin synthase that can provide a source of some biotin.

Biotin synthase (BioB) (EC 2.8.1.6) is the enzyme that catalyzes the final step in the biotin biosynthetic pathway, conversion of desthiobiotin (DTB) to biotin.

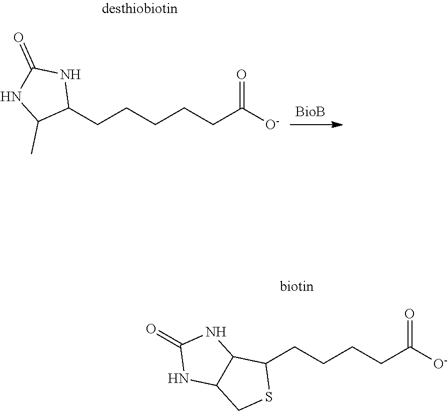 [SMILES: CC1NC(=O)NC1CCCCCC(=O)[O-] ; O=C([O-])CCCCC1SCC2NC(=O)NC21]

Biotin synthase is a member of the “radical SAM” superfamily, which is characterized by the presence of a conserved CxxxCxxC sequence motif (C, Cys; x, any amino acid) that coordinates an essential [4Fe—4S] cluster and the use of S-adenosyl-L-methionine (AdoMet or SAM) for radical generation in converting desthiobiotin to biotin.

The crystal structure of the E. coli biotin synthase in complex with SAM and desthiobiotin has been determined to 3.4 angstrom resolution (Berkovitch et al., Science, 2004, 303):76-70). The E. coli biotin synthase is a homodimer, with each monomer in the structure containing a triose phosphate isomerase (TIM) barrel with an [4Fe—4S] cluster, SAM, and an [2Fe—2S] cluster. Three of the four Fe atoms in the [4Fe—4S] cluster of the E. coli monomer chain coordinate with the 3 cysteines of the radical SAM sequence motif (Cys-53, Cys537, and Cys-60). The fourth ligand of the [4Fe—4S] cluster is an exchangeable S-adenosyl-L-methionine, which binds as an N/O chelate to the Fe through its amino-group nitrogen and carboxyl-group oxygen. The [2Fe—2S] cluster is coordinated with three cysteines (Cys-97, Cys-128, and Cys-188) and an arginine (arg-260) in the E. coli monomer. Arg-260 was also observed to interact with Ser-43, Ser-218, Ser-283, and Arg-95 in addition to the [2Fe—2S] cluster.

The [4Fe—4S] cluster (the radical SAM or RS cluster) of E. coli BioB is used as a catalytic cofactor, directly coordinating to SAM. The role of the [4Fe—4S] cofactor is to transfer an electron onto SAM, leading to formation of the 5′ deoxyadenosyl radical.

Isotopic labelling and spectroscopic studies show destruction of the auxiliary [2Fe—2S] cluster accompanies E. coli BioB turnover, indicating that it is likely a sulfur from [2Fe—2S] being incorporated into DTB to form biotin.

The E. coli biotin synthase is unable to be reactivated and is thus classified as a “suicide enzyme” since it destroys itself during turnover. The known biotin synthases have been shown to possess very poor kinetic properties.

The worldwide market for biotin as a nutritional supplement is greater than $180 million, and predicted to more than double by 2024. Currently, biotin is manufactured industrially using chemical synthesis because enzymatic synthesis or cell-based production of biotin has not been commercially viable with the previously characterized BioBs, such as the E. coli biotin synthase, due to the poor kinetic properties of the enzymes.

Thus, there is a need for a cost-competitive cell-based biotin manufacturing process to replace the current chemical syntheses.

### SUMMARY

A method of producing biotin includes contacting desthiobiotin with a Type II biotin synthase (Type II BioB) holo-protein in vitro under conditions effective to produce biotin; and recovering the biotin, wherein the Type II biotin synthase holo-protein comprises, per polypeptide chain, a first [4Fe—4S] cluster (radical SAM (RS) cluster) coordinated to a CxxxCxxC motif in the polypeptide chain and a second [4Fe—4S] cluster.

A recombinant microorganism includes a transgene encoding a polypeptide of a Type II biotin synthase, wherein a holo-protein of the Type II biotin synthase comprises per polypeptide chain a first [4Fe—4S] cluster (radical SAM (RS) cluster) coordinated to a CxxxCxxC motif in the polypeptide chain and a second [4Fe—4S] cluster.

A method for producing biotin includes cultivating the recombinant microorganism in a growth medium to produce a culture; and recovering biotin from the culture.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 presents a sequence alignment of the Blautia obeum BioB (SEQ ID NO: 1) with the E. coli K12 Biotin synthase (SEQ ID NO: 13). Solid boxes are around each of the two cys-ser swap residues in the two sequences, S-43 and C-97 in the E. coli sequence and C-52 and S-106 in the B. obeum sequence. A dotted box is around the radical SAM superfamily sequence motif CxxxCxxC. The four residues contacting the [2Fe—2S] cluster in E. coli and their corresponding residues in B. obeum are in blue.

FIG. 2 presents the UV-vis spectra of the E. coli BioB (A) and the Blautia obeum BioB (B).

FIG. 3 presents a ribbon representation of the crystal structure obtained for the B. obeum BioB, a Type II BioB.

FIG. 4 presents a ribbon structure of the active site in the Blautia obeum BioB (left) and the E. coli BioB (right).

FIG. 5 is a ribbon structure of the active site in the Blautia obeum BioB with desthiobiotin (DTB) and S-adenosylmethionine (SAM) after a dithionite (Na₂O₄S₂) soak.

FIG. 6 is a graph showing enzymatic synthesis of biotin as a function of time by two different BioB enzymes in the presence or absence of 2 mm Sulfur. Type-II BioB from Blautia obeum (triangles) and type-I BioB from Methylococcus capsulatus (circles).

### DETAILED DESCRIPTION

The inventors have discovered a new class of the radical S-adenosyl methionine (SAM) enzyme biotin synthase (BioB), referred to as “Type II biotin synthases.” Unexpectedly, Type II biotin synthases are shown to contain two [4Fe—4S] clusters per polypeptide chain in contrast to the previously identified biotin synthases, or “Type I biotin synthases”, which contain only one [4Fe—4S] cluster and one [2Fe—2S] cluster. The inventors determined X-ray crystal structures of two different Type II biotin synthases, Blautia obeum and Veillonella parvula HSIVP1, in the presence of the reaction substrates desthiobiotin (DTB) and SAM and observed the presence of two bound [4Fe—4S] clusters. One of the [4Fe—4S] clusters is a “radical SAM” (RS) cluster involved in AdoMet cleavage and ligated to a radical SAM binding motif CxxxCxxC in the primary sequence of the Type II BioB in which each of the cysteines coordinates to 3 of the Fe in the RS cluster. In the B. obeum Type II BioB sequence, the residues of the radical SAM binding motif are C-53xxxC-57xxC-60, while in the V. parvula Type II BioB sequence, the residues are C-79xxxC-83xxC-86. Three Fe atoms of the auxiliary [4Fe—4S] cluster are coordinated to three cysteine residues (C-52, C-138, and C-198 in the B. obeum Type II BioB; C-69, C-156, and C-216 in the V. parvula Type II BioB sequence. In the crystal structure, the fourth Fe in the auxiliary [4Fe—4S] cluster is coordinated to a fifth sulfur atom in close proximity to the DTB. The fifth sulfur is believed to be donated to DTB to create the biotin ring system, leaving the auxiliary [4Fe—4S] cluster intact and poised to accept a new fifth sulfur for subsequent rounds of catalysis. In vitro comparison of the biotin synthesis reaction rate of Type II BioBs and Type I BioBs unexpectedly showed that the Type II biotin synthases had a greater than 10-fold increase in enzymatic reaction rate compared to Type I biotin synthases.

A biotin synthase can be identified as a Type I or Type II biotin synthase by structural characterization, such as by X-ray crystallography, to determine the presence per polypeptide chain of a radical SAM [4Fe—4S] cluster and of an auxiliary [4Fe—4S] cluster (Type II) or an auxiliary [2Fe—2S] cluster (Type I).

Type II biotin synthases can also be identified from known biotin synthase polypeptide sequences by aligning the candidate biotin synthase sequence against a Type I BioB reference sequence, such as the E. coli biotin synthase (SEQ ID No:13), to determine if a cys-ser swap is present in the candidate sequence at the positions corresponding to E. coli S-43 and C-97. When the corresponding positions in the candidate sequence are cysteine and serine, respectively (i.e., show a cys-ser swap), the biotin synthase is a Type II biotin synthase. When the corresponding positions in the candidate sequence are serine and cysteine, respectively (i.e., no cys-ser swap), the biotin synthase is a Type I biotin synthase. Pairwise sequence alignment of the two sequences can be performed, for example, by using the BLAST program e.g. the BLASTP program (freely available online at the website of National Center for Biotechnology Information, U.S. National Library of Medicine, 8600 Rockville Pike, Bethesda, Md., 20894 USA). Multiple sequence alignment can be performed using one of the tools available online from the European Bioinformatics Institute (EMBL-EBI).

Examples of Type I Biotin synthases include those from Escherichia coli (SEQ ID No: 13); Candidatus Chloracidobacterium thermophilum B (SEQ ID No: 15; Streptomyces lydicus (SEQ ID No: 16); Paracoccus denitrificans (SEQ ID No: 17); Paracoccus denitrificans PD1222 (SEQ ID No: 18); Agrobacterium vitis (SEQ ID No: 19); Ruegeria pomeroyi (SEQ ID No: 20); Agrobacterium fabrum (SEQ ID No: 21); Wolbachia endosymbiont of Cimex lectularius (SEQ ID No: 22); Sphingomonas paucimobilis (SEQ ID No: 23); Acidithiobacillus ferrivorans (SEQ ID No: 24); Gallionella capsiferriformans (SEQ ID No: 25); Ralstonia eutropha (SEQ ID No: 26); Bordetella parapertussis (SEQ ID No: 27); Pusillimonas sp. (SEQ ID No: 28); Cenarchaeum symbiosum sp. (SEQ ID No: 29); Alicyclobacillus acidocaldarius sp. (SEQ ID No: 30); Geobacillus thermoglucosidasius (SEQ ID No: 31); Bacillus subtilis (SEQ ID No: 32); Lysinibacillus sphaericus (SEQ ID No: 33); Methylococcus capsulatus (SEQ ID No: 34); Leclercia adecarboxylata (SEQ ID No: 35); Chromohalobacter salexigens (SEQ ID No: 36); Pseudomonas spp (for example SEQ ID Nos: 37, 38, 39, 40, 41, 42, 43, 44, or 45).

Alternatively, Type II biotin synthases can be identified from known biotin synthase polypeptide sequences by aligning the candidate biotin synthase sequence against a Type II BioB reference sequence, such as the B. obeum biotin synthase (SEQ ID No:1), to determine whether the residues at the positions corresponding to B. obeum C-52 and S 106 are also a cys and a ser, respectively, indicating the candidate sequence is a Type II biotin synthase or are a ser and a cys, respectively, indicating the candidate sequence is a Type I biotin synthase.

A novel sequence can be identified as a potential biotin synthase using, for example, the online publically accessible InterPro database, which classifies protein sequences into families and predicts the presence of functionally important domains and sites using predictive models, referred to as “signatures” (See A L Mitchell, et al. (2019). InterPro in 2019: improving coverage, classification and access to protein sequence annotations. Nucleic Acids Research, January 2019; doi: 10.1093/nar/gkyI100). InterPro v. 75.0, released Jul. 4, 2019, had classified 18722 proteins as belonging to the Biotin synthase Family (IPR024177). The publically available software package InterProScan allows new sequences (protein or nucleic acid) to be scanned against InterPro's signature database for functional analysis and classification (P Jones, et al. (2014). InterProScan 5: genome-scale protein function classification. Bioinformatics, January 2014; doi:10.1093/bioinformatics/btu031). InterPro and its associated software are widely utilized by the scientific community.

Table 1 below tabulates UniProt accession numbers (entry identifiers) of Type II BioB proteins identified in the UniProt databases (The UniProt Consortium, Nucleic Acids Res. 47: D506-515 (2019)) available Aug. 27, 2019.

TABLE 1
Type II BioBs identified in UniProt
EI [symbol]
(00

| | | | | | |
|---|---|---|---|---|---|
| A0A1V5CWK7 | A0A0K2XDP6 | A0A1V5C7H2 | A0A2C8DA76 | A0A2H6FCN1 | R7D1A8 |
| A0A1B3WED7 | A0A0B0Q1S8 | A0A095YJ46 | D6Z3V5 | A0A133XRU5 | A0A0M8WHD3 |
| A0A143YJS3 | A0A099BEE7 | A0A1C0AY60 | A0A1V5DV59 | A0A2E4JQW3 | R5NVR8 |
| A0A134CP52 | A0A076F997 | L5N839 | R5IGI6 | A0A0D6I781 | Q1NIL0 |
| A0A134C0W5 | E8QRM6 | A0A1P8KQF7 | A0A1W9R7V8 | A0A1M5IR97 | Q4JWG3 |
| A0A0D8HRV4 | A0A1G3UCU1 | A0A0U0QS88 | A0A0Q9N4G1 | A0A0D6AAC8 | G5HAA7 |
| A0A143YVR3 | A0A0S4T167 | A0A1G1NY42 | A0A1J5EAV7 | A0A1F9C4P7 | A0RW96 |
| A0A132MNU4 | A0A0C2ZIG1 | A0A2N6FMP0 | A0A1G1ISA4 | A0A1C3FG73 | A0A2V1JW83 |
| A0A075GXR3 | A0A134AMA2 | A0A2G9N1R1 | A0A1B8VN82 | A0A1Y2FE74 | A0A2S6HUB6 |
| C4Z6H3 | A0A099DPP6 | A0A1Y1XPB5 | A0A0F6U2P8 | A0A1F1UY88 | A0A2M7JC52 |
| A0A2N2J6U3 | A0A087MBI2 | A0A1Q7X2K7 | A0A2H0XZ75 | B5YK85 | A0A2H6AKY5 |
| A0A2J8B5Z4 | A0A1J4YRA6 | A0A1B1U465 | A0A292SXB8 | A0A133Z539 | A0A1M6VNU1 |
| A0A1W1XIN5 | A0A1I5L5V0 | A0A1G1M3J3 | U2M7Y2 | A0A024GY50 | A0A1C3NVN8 |
| A0A1V5S9A9 | A0A0G9K380 | A0A0F8WB50 | R5SUY2 | A0A1Y4QRH2 | A0A1Y3XFQ6 |
| A0A1B1Q549 | A0A0C2ZND4 | A0A1F0MI94 | A0A2V5PWD3 | A0A1S8PM69 | A0A1Q6N9B1 |
| X8AVS6 | A0A091AJK7 | A0A2M7DUW2 | R6QDD1 | A0A1M5WJA9 | A0A1Q6MFK7 |
| A0A2S8BFK3 | A0A024BW91 | A0A1A3R1T0 | A0A1J5GNF4 | C9KNV4 | A0A1M5S325 |
| A0A0F5N7V9 | J0L607 | A0A2R6LJG1 | A0A292QS88 | A0A1F3CEL7 | A0A1H9EDE4 |
| H1HW37 | E6S4G5 | A0A0D1C6G7 | A0A0I9U4Z1 | A0A1C5YS68 | A0A1G8GT34 |
| A0A135YW82 | A0A034UA67 | A0A0J1GAW1 | A0A1F9A6J6 | A0A2N9NZE4 | Q58692 |
| X8AMW8 | D1B573 | R7HW28 | A0A0U9HLS7 | A0A1A2M4E0 | A0A1C5XQ00 |
| A0A1I6YKQ1 | A0A2D3W0K5 | N6VZE7 | A0A0E1XN76 | A0A174CSK2 | D7WAK6 |
| A0A2N2G4X3 | A0A1W1CY59 | E8LI28 | A0A091FF99 | A0A0F2NIK2 | A0A2V5NBG5 |
| A0A1V5NXU0 | A0A1T5AFT8 | A0A2N2G5X5 | T2NEJ8 | A0A099HUG1 | A0A0M2NN62 |
| A0A0T5ZJ46 | A0A0C2W160 | A0A0B2K110 | A0A2J6WM37 | A0A0D8BFK5 | A0A0E3JY29 |
| A0A2S5J2U2 | A0A099BS96 | A0A2N2ILA1 | A0A2N2GG93 | R6SBZ1 | A0A0B3WSR8 |
| A0A024JYJ5 | A0A2R6FKE1 | A0A2G3PEB6 | A0A0V8QJH6 | K1YLN3 | A0A0B2JDB2 |
| Q84HQ6 | K8GS84 | A0A0T9Y106 | A0A0F4W5W2 | A0PYU9 | A0A0A2HPH3 |
| I7L7Z5 | E6WZ23 | A0A1I4GY73 | A0A0F2PBP9 | A0A2M7N4N9 | A0A0A2EEJ6 |
| A0A059HXP1 | A0A1Y5DHV2 | A0A2M7SNI1 | B1BGB1 | A0A2A7MHX8 | A0A072XH17 |
| E7A9K8 | A0A0R2P712 | A0A1D7YSZ4 | A0A2N2K8N9 | A0A2E6TWM6 | A0A0Q0Z951 |
| A0A1W1WQN2 | A0A0C2YSC4 | R5V7R7 | A0A0P7CRR1 | A0A1T5EI95 | A0A060RA12 |
| A0A2V1IJ04 | I3Y0L2 | A0A2J0L253 | A0A0D6JVF9 | A0A1Q9JRH0 | A0A0M1JR97 |
| A0A151A8E3 | A0A2G1DFP8 | A0A2G9YN93 | A0A0D6ANN6 | A0A1G1IYJ0 | W7Y868 |
| A0A128EBE8 | A0A1V5GJR8 | A0A083W7J3 | A0A1Q3NEN1 | A0A1C5WTI4 | A0A0H3D077 |
| A0A0M4TCN1 | A0A0F6WMI8 | A0A1Q6U3M1 | A0A1M6TJY1 | A0A174IDV7 | R5W4Z3 |
| A0A099TRG3 | I2K4F4 | A0A1Q6DMZ6 | U2YG73 | A0A173R1X9 | R5HME2 |
| A0A268TEH8 | A0A1Y1UYJ5 | A0A1G1KNH3 | F3ZUN4 | A0A1H6TRI8 | H1DFD8 |
| A0A2D6CVQ3 | A0A2G2H8J3 | A0A0C1RPE3 | A0A1V6AR22 | A0A1G1IVS0 | D4RWD9 |
| A0A1W1EK82 | G0EPG6 | A0A292SMW6 | A0A1V4XE22 | A0A136Q5N3 | B0MJ71 |
| A0A2A9DR12 | A0A2N6C4K1 | A0A1F9L977 | A0A1M6FG29 | A0A1E7PK13 | B7KFJ9 |
| A0A1S6TNQ8 | A0A2G2MK79 | C0EZI5 | A0A1S1EIF2 | A0A0Q9MDN4 | A0A2N8Q376 |
| A0A1G3TF93 | A0A0Q9P351 | A0A0F0CPS2 | G4Q2V3 | R9N753 | A0A2N1UKX4 |
| A0A1F9CSX2 | A0A2H0M354 | R5H5Y2 | A0A1G9QFT3 | R7NUA3 | A0A1V6GIC5 |
| A0A1V6DM43 | A0A1H4FYM8 | B8DPP9 | A0A1H7LR99 | A0A0F2SHQ3 | A0A0C9PYB3 |
| A0A1Z8MH28 | A0A1G8N6H8 | A0A2W5IE06 | A0A1E8F019 | A0A1Z9C240 | A0A0C2HK55 |
| A0A1Y4EKU1 | A0A1G1EWP5 | A0A1F9NQV1 | A0A1F1Z655 | R7H7J7 | A0A1G7S7T7 |
| A0A1W1VCF8 | A0A1G1EUP3 | A0A1F3LCF0 | A0A1C6J2V3 | R7AZN7 | A0A011WKR3 |
| A0A0Q1CG16 | A0A1F9Z116 | A0A2U3PH22 | A0A1C0VSI5 | A0A1N7IQB4 | U6ERG2 |
| A0A1Q6KEL7 | A0A1F6QGH2 | A0A1F3DMK9 | A0A179ELW3 | Q30XZ5 | R9JSH4 |
| A0A1Q6IRF2 | A0A1E3C4H6 | A0A1C0A5W7 | A0A1B2I6P2 | A0A1C0VIK0 | K5B8K2 |
| A0A1M5SER3 | A0A0E0T4S2 | A0A174NB17 | A0A173X0F7 | K0IFX1 | K0AWM1 |
| A0A1I0DLJ1 | A0A1C5THS6 | A0A154BQR4 | A0A0U3E3S7 | J0WU76 | F5YPR5 |
| A0A1H8DGS5 | A0A174YJA8 | A0A139TQI4 | A0A0F3GQ31 | G9WZN7 | D6GQ85 |
| A0A1H5YTN4 | U1QJB8 | A0A2A2QZ62 | A0A0F2N3G3 | A0A0B5GM74 | A6UTF3 |
| A0A1H3CHI2 | Q728P5 | A0A133PJ13 | U1PBM9 | W4P3C3 | A0A2V2N392 |
| J7LKS5 | G2H5V2 | A0A0S8EBM8 | F1TF06 | R9NCA7 | C0E1U1 |
| A0A1H0VQS2 | A0A0R1MAB6 | A0A0K1RC97 | B1CC62 | A7I1A0 | A0A2W6BQE0 |
| I3DBT0 | A0A0M8YM96 | A0A0M6WZ28 | B0NDK1 | A0A2D5MV29 | A0A2H6JR58 |
| A0A1G5CW90 | A0A0C3NHC3 | A0A0K1HH81 | A0A2V3I2Y5 | A0A2V1IYS8 | A0A2E5EIA1 |
| C9REY2 | A0A0B5QPC1 | A0A0H3J483 | A0A1G9DZ47 | A0A2U3QIG8 | A0A2H5V830 |
| A0A1G1G1H3 | A0A0A2DWE4 | A0A0A1VZF1 | A0A2M9A3C1 | A0A2K2TPB3 | A0A267MCU5 |
| A0A2N6T4P7 | A0A1Q2HUG2 | A0A0F2PT43 | A0A2H6IEI9 | A0A1I0MB16 | A0A1I3KM86 |
| A0A1C5XMQ1 | V6V4Y1 | A0A0F2JHU2 | A0A2G4JEH2 | A0A1G4VN44 | A0A1V4YBH8 |
| A0A1C2BTA1 | R6ZBB1 | A0A0D0SD75 | A0A0F5YJ99 | A0A1W1V284 | A0A1M6QPC8 |
| A0A174SP97 | R6Z1M9 | A0A072X1Q9 | A0A2N9NUD1 | A0A073CBN5 | A0A1M6C0B6 |
| A0A127EJD2 | A0A0U0W4C1 | R6SVR5 | A0A1V0LXM7 | A0A1V5CD13 | A0A1L8CV90 |
| A0A0S8AQV8 | M1NK09 | R6DF74 | A0A1T5IAN7 | A0A1M5U4D1 | A0A1K2HCP7 |
| A0A1X9L5P4 | F6BCQ0 | A0A2D7B5E8 | A0A1H6FVB2 | A0A1M4UV64 | A0A1U7NBI3 |
| A0A0F7F7I0 | C3JD21 | R5AK74 | A0A1T4JY46 | A0A1M4U5J4 | A0A1H7QJ49 |
| A0A095ZPM2 | A0A068MT00 | L0K509 | A0A1Q7F628 | A0A1J5EHR7 | A0A1G5S281 |
| A0A069S508 | D7GUL2 | A0A1Q2TW91 | A0A1Q6N581 | A0A1V2IYS0 | A0A1F7RMK6 |
| V2RKH3 | A0A2H6FI69 | E8RJ39 | A0A166UB95 | A0A1H6WJJ1 | A0A1F4TLE9 |
| S7HQF5 | A0A2X4TU61 | E0E4W1 | A0A1M6VVV4 | A0A1J1KDZ2 | A0A1D9FMU9 |
| R7P9Z0 | A0A2G4EUZ3 | D6STN3 | A0A1M5D202 | A0A1H1BQZ4 | A0A1C0ANZ9 |
| A0A095WKQ8 | A0A255JG18 | A0A2P8R0N3 | A0A1K1VYJ1 | A0A1H2IGN9 | A0A173VFD2 |
| A0A2X1MM29 | A0A1V8XA55 | A0A0E7TCZ8 | A0A1I5J4K8 | A0A1H0VDZ7 | K9W277 |
| A0A062WP45 | A0A1S1RTM4 | F1TJ48 | K9TEM4 | A0A1G9YN66 | A0A140LDL7 |
| A0A2S4GKH3 | A0A1V5VYY4 | A0A2E7QKP7 | A0A2E8Y4N4 | A0A1G1JQM2 | A0A135L3D5 |
| A0A2N2EFC6 | A0A1U7LCS9 | A0A259UBE5 | A0A1F8ZN31 | A0A1G1HBE6 | A0A126R1R6 |
| A0A2L2XBI5 | A0A1Q6TV57 | A0A239REM9 | A0A1F6QHF7 | A0A1F3CBP0 | A0A1P8YEE0 |
| A8L2N0 | A0A1Q6EEX4 | A0A1V4W6U9 | A0A1C5LFU2 | A0A1C6DJG1 | A0A0M6WDP1 |
| A0A2H6FTM4 | A0A1M4VFY8 | A0A1T4JQU5 | A0A1G8W3Z5 | A0A1C6A6Y4 | A0A0L6VZ52 |
| A0A1Y4RDV7 | A0A0M1VT35 | A0A1S8NJS8 | A0A1B3WNV7 | A0A1B9BKQ3 | A0A0A2I5P5 |
| A0A1Y4GGU9 | A0A1H3GD71 | A0A1M6SW20 | A0A173UAU4 | A0A2D5VZD3 | A0A081RPG1 |
| A0A1I6JWQ1 | A0A1H0UVB6 | A0A1J5ED84 | A0A143ZTV7 | A0A101G5E6 | D5RA42 |
| A0A1E5QZA0 | A0A1H0DLV7 | A0A0D8ZXI8 | A0A143YQY9 | A0A1G9Z1A7 | A0A060HH48 |
| A0A1I3PLD4 | A0A1G6BQY0 | A0A1I7JIE2 | A0A139CP83 | A0A0Q1BN59 | A0A037Z8Q0 |
| A0A031WBV1 | F4XL14 | A0A076LDY0 | C8PJG8 | A0A194AIU9 | A0A015QZI3 |
| R5Z3S3 | A0A0N7HBZ6 | A0A069SD64 | B7AVK6 | A0A173YRZ5 | A0A078QIB9 |
| L8TW02 | A0A0F5J5I5 | A0A017H7M1 | A8ZUS9 | A0A143B7Z4 | A0A139KCK9 |
| A0A1I6SPN6 | A0A0B2YRB4 | R5P1Q6 | A1HTZ7 | A0A0K8JBT7 | A0A078RYC7 |
| G9S1I7 | A0A085LB86 | E6U6V0 | A0A2T4UFU1 | A0A0K2J8E1 | A0A081U5F2 |
| F8ANR0 | A0A075GJW4 | E5Y1J5 | A0A2N2H7T9 | A0A0J6X060 | A0A1I1YYK3 |
| F7NGP3 | V1CM26 | D4LRD1 | A0A2G6MQT7 | A0A0E1EKB5 | A0A015T5U0 |
| D9QSE3 | R5D3Z0 | C3WCI1 | A0A2G6BP80 | A0A0D5A6X7 | A0A1H8V0B0 |
| B5W204 | H1D0T6 | B8FHQ1 | A0A259ULM4 | A0A0A2G503 | A0A0P0GXW3 |
| D3E0W3 | E0QJM7 | A0A1H6FM91 | A0A1W9WE89 | A0A075K8I0 | A0A1G1KWV8 |
| C7P5F1 | A0A2T0BA29 | A6UPN7 | A0A1V5CUR5 | A0A069ZL73 | B0NM25 |
| B0TE53 | A0A2N6EGL0 | A1AN77 | A0A1T4KZ66 | F7V873 | E4VS48 |
| A0A2S1QHF4 | A0A2L1CC74 | A0A2M9DV31 | A0A1Q6SQE3 | D8FFI4 | H3KD46 |
| A0A063ZM26 | A0A2J6J8Y6 | A0A2H1EI92 | A0A1Q6R5K1 | D6KKW8 | A0A1Q6H8L9 |
| A0A1D8U354 | M0CMX0 | A0A2E9KWI4 | A0A1M7JQ57 | A0A2N7Q2H3 | A0A174DIH2 |
| A0A2K9EI95 | A0A1Z9ENZ0 | A0A250LXD5 | A0A1M6JZJ3 | A0A2N5RIB9 | A0A0J9FH94 |
| A0A2J6IW04 | A0A1T4V4Z4 | A0A1V5CND6 | A0A1L7CQ75 | A0A2N2HSV7 | J9FCJ8 |
| A0A2G6FPU3 | A0A1S1V521 | A0A1T4Q009 | A0A1G3PU59 | A0A269TIG4 | A0A0T5ZVM3 |
| A0A1D9G0Z8 | A0A1Q7VWC1 | A0A1Q8R371 | A0A1G1HB24 | A0A2E7M6F8 | A0A1Y4VKY2 |
| A0A1W2CUD1 | A0A1M4W854 | A0A1M6DUM6 | A0A1G1DZG3 | A0A1V5CCB6 | A0A1C7H0D9 |
| A0A1V5V3P3 | A0A1L3GI92 | A0A1M6B8E4 | A0A1F8X4Z6 | A0A1T4QEK3 | A0A120A0C9 |
| A0A1V4WGF8 | A0A1H7Y1I2 | A0A1L5F8K8 | A0A1F5J1X8 | A0A1H8V245 | I9RU87 |
| A0A2K8WKX4 | A0A1G5D8F2 | A0A1I4NE78 | A0A1F4T7D6 | A0A1G4Q316 | A0A0J0UWH2 |
| A0A1Q6IKK3 | A0A1G2X174 | A0A1H6H7N9 | A0A1F4SV21 | A0A1G1DD30 | A0A1Q6GAC7 |
| A0A1M6JE75 | A0A1G1GE54 | A0A1G9PY58 | A0A1F2UA73 | A0A173VQD6 | A0A173UJW9 |
| A0A1I7HR32 | A0A1G1EAF5 | A0A1G1K9I2 | A0A1C5QZ15 | A0A173TRX3 | A0A0F5N130 |
| A0A1G9R6P6 | A0A1F9ILG8 | A0A1G0MI31 | A0A0M2UPQ3 | A0A135YR02 | D6D4S8 |
| A0A1G5AY05 | A0A1F9AG32 | A0A1F8YEK4 | A0A0B5FH10 | A0A0G3H441 | A0A0B0ELS7 |
| A0A1G3QPW2 | A0A1F4Q544 | H0E2Y4 | A0A0B5B9L2 | A0A024QNU8 | J9C8L1 |
| A0A1G1JWQ9 | A0A1E8GCG7 | A0A1F8WZX5 | A0A096BGX8 | R6VIU8 | A0A0A2FLX2 |
| A0A1G1FAH3 | A0A1E7IP74 | A0A1F5J3R3 | D3F023 | O67104 | A0A0A2EBK0 |
| A0A285N827 | A0A1B1SDC5 | A0A1F4RG15 | U7L975 | C9LND7 | R7CV06 |
| A0A1F9CN20 | A0A173WZR4 | A0A1E7GTV1 | Q3ANX4 | A4SGW2 | Q1Q6F4 |
| A0A1V6IYQ7 | A0A0U1CM78 | A0A0M4CX24 | A0A2I0NEI1 | A0A2E5AU64 | G9YJX7 |
| A0A1F8XD66 | A0A0T6ACW6 | B4VWP6 | A0A2G9XL20 | A0A1X7IQ62 | D3SLC8 |
| A0A1F7SCK4 | A0A0S6VUE2 | A0A0F2J0U2 | A0A2G6MH89 | W4USZ4 | B2V8G9 |
| A0A1F4U6K9 | A0A0N8W7I9 | A0A2W5ZIQ1 | A0A1Z8TEE5 | A0A1V5UMF9 | A0A2U1B9V4 |
| A0A1F1E0Y7 | A0A0L0W7W7 | A0A0C1B953 | A0A1X4KH66 | F3PHX7 | A0A2P6UT82 |
| A0A1E7IDD1 | A0A0A2TMI4 | A0A0A8WT92 | A0A1V4S2G2 | A0A1V4VIS3 | A0A2E8DTW3 |
| A0A1E3XCS7 | A0A097ICW8 | A0A0A7HIR5 | A0A1M6ST89 | A0A1Q6I1X6 | A0A2C8D7D9 |
| A0A154BLS9 | A0A097AQ29 | A0A075HMN8 | A0A1J5DDR5 | D7IG61 | A0A1W9PUL8 |
| A0A128A2Z0 | A0A095Z4F4 | A0A069RGJ6 | A0A1G6KV49 | E5VCK1 | A0A1T4NLA6 |
| A0A101DJ08 | R4WAG2 | W8T3G6 | A0A1G3PKX3 | J9C364 | A0A1I3CCE1 |
| M0KPK0 | A0A084JCY6 | U7V9V4 | A0A1G0M705 | A0A108T4A7 | A0A1D8D5Y1 |
| A0A1C5M5A8 | A0A095Y1Z3 | A0A1Y4NZY2 | | | |
| A0A134CNR2 | I9LGG0 | A0A1V5RI74 | | | |
| A0A0U1L020 | H7EKD9 | | | | |
| A0A0K8JBM8 | C0XTQ8 | | | | |
| A0A0G4K3B5 | B4S690 | | | | |
| A0A0G3GSC5 | A0A2G6MCM1 | | | | |
| R6X2R8 | A0A285NQ98 | | | | |
| F4KLX8 | A0A1W7QR93 | | | | |
| A5N8V7 | A0A1Q3RLG5 | | | | |
| A1BCQ5 | A0A1G3AN56 | | | | |
| A0A2T5CD15 | A0A165M764 | | | | |
| A0A2E3B6N8 | A0A100YT45 | | | | |
| A0A1Q9UJ16 | A0A0S7X7B2 | | | | |
| A0A1M6SIM1 | A0A0M6WF21 | | | | |
| A0A1M6QX09 | A0A059MPY8 | | | | |
| A0A1I0FX15 | I5ASY6 | | | | |
| A0A1G5UWP5 | C7NAZ6 | | | | |
| A0A1G0N2M6 | A0A2E8Q1C0 | | | | |
| A0A1B8WWJ1 | A0A1U7MC52 | | | | |
| A0A174A8F5 | A0A1B7LGF6 | | | | |
| A0A101F5Y6 | A0A174NU23 | | | | |
| A0A0E3N7L8 | A0A134CAY9 | | | | |
| A0A075KCK0 | A0A075ULR7 | | | | |
| A0A072Z4B1 | B3EPW2 | | | | |
| C1DTU4 | A0A222P925 | | | | |
| A0A2N2JTN2 | A0A1T4L1X1 | | | | |
| A0A2G6JJQ2 | A0A1Q6RAX9 | | | | |
| A0A2G6HMY4 | A0A1M5X971 | | | | |
| A0A2E0XJL8 | A0A1H1U0T6 | | | | |
| A0A2D5ZRW3 | A0A0M5L1L3 | | | | |
| A0A1Y0HDJ6 | A0A0B4ELR0 | | | | |
| A0A1U7MJP4 | A0A095ZGA0 | | | | |
| A0A1Q6P7K3 | A0A1D8BVB3 | | | | |
| A0A1G7QTU3 | A0A0C1E105 | | | | |
| A0A024LV64 | A0A087S7G1 | | | | |
| Q6AK48 | L8AF29 | | | | |
| C2MAL4 | E2ZBH3 | | | | |
| C0QR28 | A0A2P8EIF3 | | | | |
| A0A2S0WBH5 | A0A133S5G0 | | | | |
| A0A1M5B3E3 | A0A109CGK9 | | | | |
| A0A101JU82 | A0A0G2Z468 | | | | |
| A0A0K1FII1 | A0A024H1Y0 | | | | |
| A0A0F2R476 | F5RL49 | | | | |
| A0A0D8IEV9 | A0A2H0LMU9 | | | | |

[symbol]

indicates data missing or illegible when filed

A method of producing biotin is disclosed. The method can comprise contacting desthiobiotin with a Type II biotin synthase (Type II BioB) holo-protein in vitro under conditions effective to produce biotin; and recovering the biotin.

The Type II BioB can be selected to provide desired characteristics in production of biotin, such as kinetic efficiency, absolute production, and scalability of the process. For example, the Type II biotin synthase can be a Blautia sp. biotin synthase, a Clostridium sp. biotin synthase, a Bacteroides sp. biotin synthase, a Porphyromonas sp. biotin synthase, a Veillonella sp. biotin synthase, a Cyanothece sp. biotin synthase, an Akkermansia sp. biotin synthase, or a combination thereof. Preferably, the type II the biotin synthase can be a Blautia obeum biotin synthase (SEQ ID NO:1), a Clostridium sp. HMSC19B10 biotin synthase (SEQ ID NO:2), a Bacteroides caccae (ATCC 43185) biotin synthase (SEQ ID NO:3), a [Clostridium] spiroforme DSM 1552 biotin synthase (SEQ ID NO:4), a Porphyromonas gingivalis (strain ATCC BAA-308/W83) biotin synthase (SEQ ID NO:5), a Bacteroides cellulosilyticus biotin synthase (SEQ ID NO:6), a Clostridium perfringens biotin synthase (SEQ ID NO:7), a Clostridium thermocellum biotin synthase (SEQ ID NO:8), a Veillonella parvula HSIVP1 biotin synthase (SEQ ID NO:9), a Cyanothece sp. (strain ATCC 51142) biotin synthase (SEQ ID NO:10), a Porphyromonas gingivalis (strain ATCC 33277) biotin synthase (SEQ ID NO:11), an Akkermansia muciniphila (strain ATCC BAA-835/Muc) biotin synthase (SEQ ID NO:12), or a combination thereof.

Contacting the desthiobiotin with the Type II biotin synthase holo-protein can occur in a cell-free system.

The conditions effective to produce biotin can include S-adenosylmethionine (SAM), NADPH, and one or more polypeptides to mediate transfer of an electron from NADPH to the radical SAM [4Fe—4S] cluster. Polypeptides to mediate transfer of an electron from NADPH to the radical SAM [4Fe—4S] cluster include a flavodoxin/ferredoxin-NADP reductase; a pyruvate-flavodoxin/ferredoxin oxidoreductase; a flavodoxin; a ferredoxin; or a combination thereof. In a cell-free system, amounts of the Type II BioB, DTB, SAM, and NADPH and the electron transfer polypeptides can be selected to optimize production of biotin.

Recovering the biotin from the reaction can be performed by any suitable method. For example, the enzymes can be precipitated from the reaction, and the biotin in the supernatant can be absorbed on active carbon, then eluted and purified further with an ion exchange resin. Alternatively, the reaction can be applied directly to an ion exchange resin and, after the elution, the biotin can be recrystallized from a mixture of alcohol and water.

Biotin can be produced using recombinant microorganisms, e.g., bacterial cells such as recombinant E. coli cells, expressing a Type II BioB by culturing the recombinant microorganism in a culture medium suitable for supporting growth as well as comprising a carbon source suitable for the biosynthesis of biotin.

The method for producing biotin can comprise: culturing a recombinant microorganism comprising a transgene encoding a polypeptide of a biotin synthase comprising two (4Fe—4S) clusters per polypeptide chain (Type II BioB) in a growth medium to produce a culture; and recovering biotin produced by the culture.

The culture medium and the temperature and time for cultivation of the recombinant microorganism are selected to optimize production of biotin.

The growth medium used in the method for producing biotin can comprise a carbon source selected from desthiobiotin, glucose, maltose, galactose, fructose, sucrose, arabinose, xylose, raffinose, mannose, lactose, or any combination thereof.

The pH of the culture medium can be about 5.0 to 9.0, preferably 6.5 to 7.5. The cultivation temperature can be about 10 to 400° C., preferably 26 to 30° C. The cultivation time may be about 1 to 10 days, preferably 2 to 7 days, more preferably about 2 to 4 days (48 to 96 hours). During cultivation, aeration and agitation usually give favorable results.

Recovering the biotin from the culture medium can be performed by any suitable method. For example, the cells can be removed from the culture medium, the desired product in the filtrate can be absorbed on active carbon, then eluted and purified further with an ion exchange resin. Alternatively, the culture filtrate can be applied directly to an ion exchange resin and, after the elution, the desired product can be recrystallized from a mixture of alcohol and water.

The Type II biotin synthase can be expressed in a heterologous microorganism. The expressed Type II biotin synthase can be purified and used in a cell-free system to produce biotin. Alternatively, the microorganism expressing the Type II biotin synthase can be cultured under conditions permitting biotin production. The amount of recovered biotin can be increased compared to amount of recovered biotin from culturing the same microorganism that does not express the biotin synthase. The microorganism can also comprise transgenes for expression of other enzymes or regulatory moieties involved in the biotin synthetic pathway to further enhance biotin production or in assembling Type II BioB holo-protein. For example, the microorganism can also comprise the plasmid pDB1282 containing the isc operon from Azotobacter vinelandii, which encodes the proteins IscS, IscU, IscA, HscB, HscA, and Fdx. The plasmid is inducible with arabinose and confers antibiotic resistance to ampicillin. Additionally or alternatively, the microorganism can also comprise the plasmid pPH151 containing the suf operon from E. coli, encoding the proteins SufA, SufB, SufC, SufD, SufS, and SufE, which is inducible with IPTG and confers antibiotic resistance to chloramphenicol.

Exemplary microorganisms for expressing a Type II BioB or for producing biotin include bacteria, yeast, and filamentous fungi.

Exemplary bacteria for expressing a Type II BioB or for producing biotin include a species of Escherichia, Bacillus, Brevibacterium, Burkholderia, Campylobacter, Corynebacterium, Serratia, Lactobacillus, Lactococcus, Acinetobacter, Acetobacter, Pseudomonas, and Vibrio natriegens; preferably the bacterium is a species of Escherichia or Corynebacterium; for example Escherichia coli or Corynebacterium glutamicum.

Exemplary yeast for expressing a Type II BioB or for producing biotin include Saccharomyces cerevisiae, Pichia pastoris, Hansenula polymorpha, Komagataella sp., Kluyveromyces lactis, and Yarrowia lipolytica.

Examples of filamentous fungi for expressing a Type II BioB or for producing biotin include Aspergillus, Trichoderma, Penicillium and Rhizopus species. Preferably the fungi is Trichoderma reesei, Aspergillus niger, or Aspergillus oryzae.

A recombinant microorganism comprising a transgene encoding a polypeptide of a biotin synthase, wherein the biotin synthase comprises two (4Fe—4S) clusters per polypeptide chain (Type II BioB) is disclosed. The Type II BioB transgene can be operably linked to a constitutive promoter.

Optionally, the recombinant microorganism may further comprise one or more additional transgenes encoding polypeptides that catalyze additional steps in the biotin biosynthetic pathway. An increase in the levels of those polypeptides that catalyze steps in the biotin biosynthetic pathway enhances the synthesis of both intermediates in the biotin pathway, and the end product of the pathway (biotin) in the cell.

The polypeptides that are encoded by the additional transgenes in the recombinant microbial cell, and whose activity serves to enhance the synthesis of both intermediates and products of the biotin pathway, can include a polypeptide having SAM (S-adenosylmethionine)-dependent methyltransferase activity (BioC); a polypeptide having 7-keto-8-aminopelargonic acid (KAPA) synthase activity (BioF); a polypeptide having 7,8-Diaminopelargonic Acid (DAPA) Synthase activity (BioA); or L-lysine: 8-amino-7-oxononanoate aminotransferase (BioK); a polypeptide having Desthiobiotin (DTB) Synthetase activity (BioD); a polypeptide having Pimeloyl-[acyl-carrier protein] methyl ester esterase (BioH); a polypeptide having 6-carboxyhexanoate-CoA ligase activity (BioW) or a combination of the foregoing.

The transgene encoding BioB together with one or more additional transgenes encoding polypeptides that catalyze additional steps in the biotin pathway, are located in the genome of the recombinant microorganism, either integrated into the chromosome or on a self-replicating plasmid. The transgenes encoding BioB and one or more enzymes in the biotin pathway (BioABFCD and H or W) can be present in the genome within one or more operon.

The promoter driving expression of the transgene encoding BioB together with one or more additional transgenes is preferably a non-native promoter, which can be a heterologous constitutive-promoter or an inducible-promoter. Examples of a suitable heterologous constitutive promoter include members of the apFab family [SEQ ID Nos: 46-48] while a suitable inducible promoter includes: pBad (arabinose inducible [SEQ ID No: 49] and Lacl [SEQ ID No: 50]. Suitable terminators include members of the apFAB terminator family including [SEQ ID No:51-53]. The selected promoter and terminator can be operably linked to the coding sequence for BioB. The selected promoter and terminator can also be operably linked to the coding sequence for BioB and to the coding sequence of the polypeptides of BioC, BioD, BioA, BioF, BioW BioH, or a combination thereof.

The terms “polypeptide,” “peptide”, and “protein” are used interchangeably herein to refer to a molecule formed from the linking, in a defined order, of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

An “apo-protein” refers to a BioB polypeptide chain without its complement of Fe—S clusters, while a “holo-protein” refers to a BioB polypeptide chain refers to a BioB polypeptide chain with its complement of Fe—S clusters.

The terms “isolated” or “purified”, used interchangeably herein, refers to a nucleic acid, a polypeptide, or other biological moiety that is removed from components with which it is naturally associated. The term “isolated” can refer to a polypeptide that is separate and discrete from the whole organism with which the molecule is found in nature or is present in the substantial absence of other biological macro-molecules of the same type. The term “isolated” with respect to a polynucleotide can refer to a nucleic acid molecule devoid, in whole or part, of sequences normally associated with it in nature; or a sequence, as it exists in nature, but having heterologous sequences in association therewith; or a molecule disassociated from the chromosome. Purity and homogeneity are typically determined using analytical chemistry techniques, for example polyacrylamide gel electrophoresis or high performance liquid chromatography. A protein that is the predominant species present in a preparation is substantially purified. In some embodiments, the term “purified” means that the nucleic acid or protein is at least 85% pure, specifically at least 90% pure, more specifically at least 95% pure, or yet more specifically at least 99% pure

The term “recombinant” can be used to describe a nucleic acid molecule and refers to a polynucleotide of genomic, RNA, DNA, cDNA, viral, semisynthetic, or synthetic origin which, by virtue of its origin or manipulation is not associated with all or a portion of the polynucleotide with which it is associated in nature. The term “recombinant” as used with respect to a protein or polypeptide can refer to a polypeptide produced by expression of a recombinant polynucleotide. In general, the gene of interest is cloned and then expressed in a transformed organism, by a suitable method. The host organism, or recombinant organism, expresses the foreign gene to produce the protein under expression conditions.

An expression vector comprising a polynucleotide encoding a Type II BioB polypeptide is also disclosed.

The term “vector” means a nucleic acid sequence to express a target gene in a host cell. Examples include a plasmid vector, a cosmid vector, a bacteriophage vector, and a viral vector. Examples of viral vectors include a bacteriophage vector, an adenovirus vector, a retrovirus vector, and an adeno-associated virus vector.

For example, the vector may be an expression vector including a membrane targeting or secretion signaling sequence or a leader sequence, in addition to an expression control element such as promoter, operator, initiation codon, termination codon, polyadenylation signal, and enhancer. The vector may be manufactured in various ways depending on the purpose. An expression vector may include a selection marker for selecting a host cell containing the vector. Further, a replicable expression vector may include an origin of replication

The term “recombinant vector” or “expression vector” means a vector operably linked to a heterologous nucleotide sequence for the purpose of expression, production, and isolation of the heterologous nucleotide sequence. The heterologous nucleotide sequence can be a nucleotide sequence encoding all or part of a Type II BioB.

The recombinant vector may be constructed for use in prokaryotic or eukaryotic host cells. For example, when a prokaryotic cell is used as a host cell, the expression vector used generally includes a strong promoter capable of initiating transcription (for example, pLλ promoter, trp promoter, lac promoter, tac promoter, T7 promoter), a ribosome binding site for initiating translation, and a transcription/translation termination sequence. When a eukaryotic cell is used as a host cell, the vector used generally includes the origin of replication acting in the eukaryotic cell, for example f1 origin of replication, SV40 origin of replication, pMB1 origin of replication, adeno origin of replication, AAV origin of replication, or BBV origin of replication, but is not limited thereto.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the nucleic acid sequences being linked are contiguous and, where necessary to join two protein coding regions, contiguous and in reading frame.

A “transgene” is an exogenous gene that has been introduced into the genome of a bacterium by means of genetic engineering. In the context of the present invention, said genome includes both chromosomal and episomal genetic elements.

The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity can be calculated as ((Nref−Ndif) 100)/(Nref), wherein Ndif is the total number of non-identical residues in the two sequences when aligned and wherein Nref is the number of residues in one of the sequences. Readily available computer programs can be used to aid in the analysis of sequence identity. Sequence identity calculations are preferably automated using for example the BLAST program e.g. the BLASTP program, available on the internet from the National Center for Biotechnology Information. Multiple sequence alignment (MSA) can be performed with one of the programs for MSA available on the internet from, for example, the European Bioinformatics Institute. Two nucleic acid or two polypeptide sequences are “substantially identical” to each other when the sequences exhibit at least about 50%, specifically at least about 75%, more specifically at least about 80%-85%, at least about 90%, and most specifically at least about 95%-98% sequence identity over a defined length of the molecules.

The numbers of substitutions, insertions, additions or deletions of one or more amino acid residues in the polypeptide as compared to its comparator polypeptide can be limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3 proline; group 4: Phenylalanine, Tyrosine, Tryptophan; Group 5: Aspartate, Glutamate, Asparagine, Glutamine.

An “endogenous gene” a gene in a bacterial cell genome that is homologous in origin to a host bacterium (i.e. a native gene of the host bacterium). The endogenous gene may be genetically modified using tools known in the art whereby the genetically modified endogenous gene encodes a mutant polypeptide whose amino acid sequence differs at one or more position from the polypeptide encoded by the parent endogenous gene from which it was derived.

The term “genome” is the genetic material present in a cell or organism comprising all of the information needed to build and maintain that cell or organism. Herein, genome includes the genetic material in both chromosome(s) and plasmid(s) present within the cell or organism.

A “native gene” is an endogenous gene in a bacterial cell genome, homologous to host bacterium.

A “non-native promoter”, in the context of a recombinant microorganism, is a promoter that is operably-linked to a gene or transgene in the microorganism, which would not be found operably-linked to the gene or transgene in the microorganism cell found in nature.

The following examples are merely illustrative of the methods and compositions disclosed herein and are not intended to limit the scope hereof.

### EXAMPLES

### Ultraviolet-Visible (UV-Vis) Spectrophotometry

UV-vis spectrophotometry was performed using Agilent Technologies 8453. In general, UV-visible spectra at 10-20 μM protein by diluted concentrated stocks in size exclusion buffer

### X-Ray Crystallography

Diffraction-quality crystals were obtained by sitting-drop vapor diffusion at 20° C. in an anaerobic chamber maintained at <0.1 ppm oxygen (MBraun, Stratham, N.H.). Drops of 0.4 μL TEV-cleaved protein solution at 20 mg/mL in 25 mM HEPES, pH 7.5, 0.7 mM SAM, and 0.7 mM DTB were mixed with 0.4 μL precipitant (0.1 M ammonium acetate, 0.1 M Bis-tris, pH 5.5, 17% polyethylene glycol 10,000) and equilibrated against a solution of 0.5 M LiCl. Diffraction data were collected at the Advanced Photon Source (Argonne National Laboratory, Argonne Ill.) and the structure was solved by SAD phasing using the intrinsic Fe absorption.

### Example 1. Blautia obeum Biotin Synthase-Like Protein

Blautia obeum (formerly Ruminococcus obeum) is a species of anaerobic, gram-positive bacteria found in the human gut that was identified as having a biotin synthase-like protein (UniProtKB A5ZUL4) in its genomic sequence.

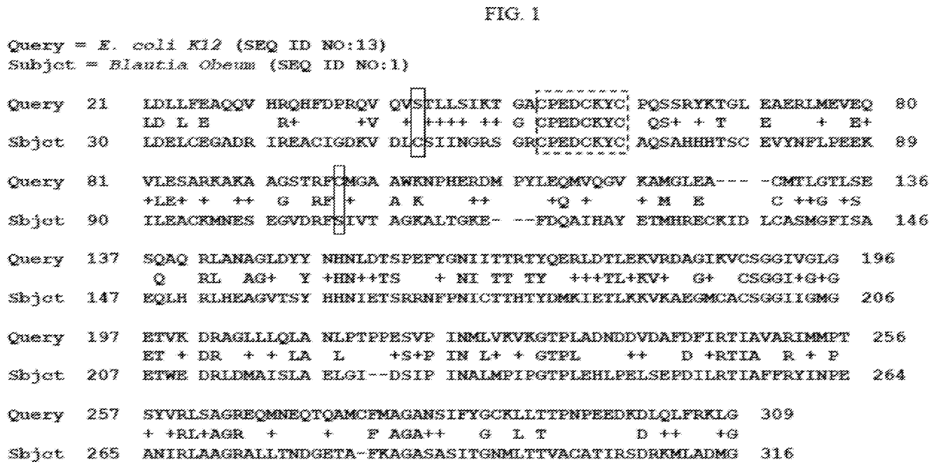

Alignment of the B. obeum amino acid sequence with the E. coli BioB sequence showed that the B. obeum sequence was roughly 50% identical to that of the E. coli BioB sequence as shown in FIG. 1. The alignment in FIG. 1 shows that in the B. obeum sequence a serine (Ser 106) is present at the position corresponding to the E. coli BioB cysteine-97. E. coli BioB cysteine-97 binds an Fe atom in the auxiliary [2Fe—2S] cluster, however the serine in the B. obeum sequence can no longer bind an Fe atom in the cluster. The alignment in FIG. 1 also shows serine-43 in the E. coli BioB sequence is a cysteine (Cys-52) in the corresponding position in the B. obeum sequence. This new arrangement of cysteine residues in the active site of the B. obeum Type II BioB sequence compared to the exemplary Type I BioB sequence of E. coli is referred to herein as “the cysteine-serine swap” and is characteristic of all Type II biotin synthases identified to date. As discussed further below, this new arrangement of cysteine residues in the active site of the Type II biotin synthases allows the Type II BioBs, such as the B. obeum BioB, to bind an auxiliary [4Fe—4S] cluster, rather than the auxiliary [2Fe—2S] cluster characteristic of the Type I BiioBs.

The B. obeum sequence was expressed and purified from BL-21(DE3) cells containing the pPH151 plasmid. The transformants were selected on an LB/agar plate containing 50 μg/mL kanamycin and 34 μg/mL chloramphenicol. A single colony was used to inoculate 20 mL of LB overnight culture containing the above antibiotics. 20 mL of the overnight culture was used to inoculate 2 L of LB media housed in a 2 L PYREX® media bottle. Cultures were grown with constant aeration using a sparging stone attached to a pressurized, 0.22 μm filtered air source all in a water bath maintained at 37° C. After 5 hr, aeration was stopped and the culture was placed in an ice bath for 1 hr. The culture was returned to a 22° C. water bath and light aeration was resumed. After 5 min, cysteine and IPTG were added to a final concentration of 600 and 500 μM, respectively. The culture was grown at 22° C. for ˜20 hr before being harvest by centrifugation at 10,000×g. Cell pellets were flash frozen and stored in liquid N2 until purification. All subsequent steps were carried out in an MBraun anaerobic chamber maintained at <0.1 ppm oxygen (MBraun, Stratham, N.H.). In a typical purification, ˜30 grams of cell paste was resuspended in 30 mL of lysis buffer containing 50 mM HEPES, pH 7.5, 300 mM KCl, 4 mM imidazole, 10 mM 2-mercaptoethanol (BME), 10% glycerol, and 1% Triton-X305. The resuspension was subjected to 50 rounds of sonic disruption (80% output, 3 s pulse on, 12 s pulse of) at 4° C. The lysate was cleared by centrifugation at 4° C. for 1 hr at 15,000×g. The supernatant was loaded with an ÄKTA express FPLC system onto a 5 mL fast-flow HisTrap™ column (GE Healthcare Life Sciences) equilibrated in lysis buffer lacking Triton-X305. The column was washed with 10 column volumes of lysis buffer before elution with 5 mL of buffer containing 50 mM HEPES, pH 7.5, 300 mM KCl, 300 mM imidazole, 10 mM BME, and 10% glycerol. The fractions containing protein, based on absorbance at 280 nm, were pooled and reconstituted with Fe and sulfur as previously described. The reconstituted proteins were then passed over a HiPrep 16/60 Sephacryl S-200 HR column equilibrated in 20 mM HEPES, pH 7.5, 300 mM KCl, 5 mM DTT, and 10% glycerol. The proteins were concentrated to ˜1 mL with a vivaspin 20 concentrator (Sartorius Stedium Biotech). The protein concentration was estimated by A280 using the extinction coefficient calculated based on the targets corresponding amino acid sequence.

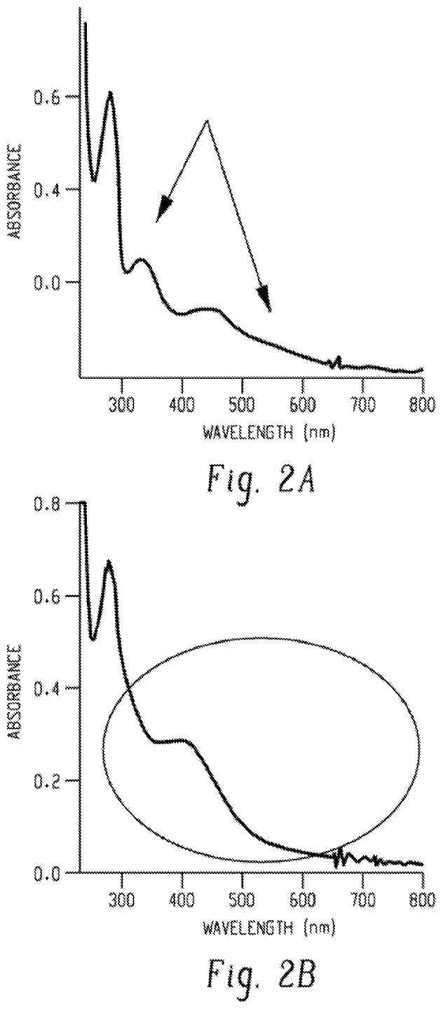

As shown in FIG. 2, the UV-vis spectra of the B. obeum protein (B) differed from that of the E. coli BioB (A). The E. coli BioB spectrum includes features typical of the [2Fe—2S] cluster in the 330 nm to 550 nm wavelength region. However the B. obeum protein lacked those features.

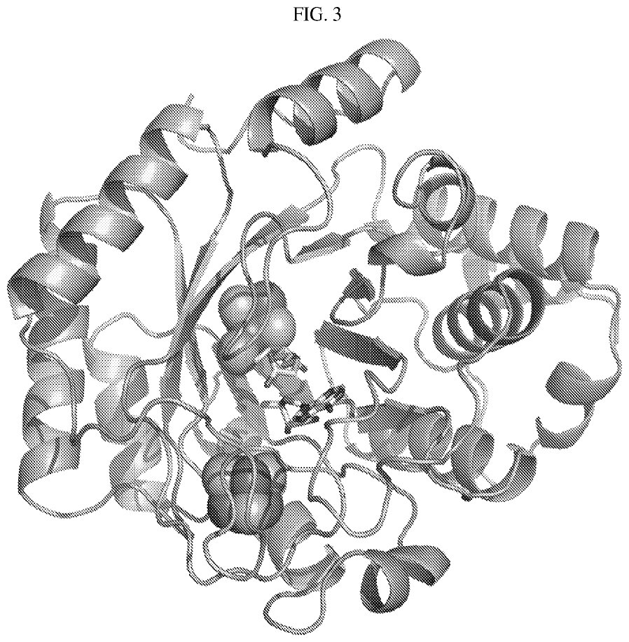

Crystals of the B. obeum protein were grown and the structure was determined by X-ray crystallography. A ribbon representation of the structure determined for the B. obeum protein in the presence of DTB and SAM is shown in FIG. 3. The B. obeum protein acts as a monomer in solution and the crystal packing. In contrast to the E. coli BioB, the B. obeum BioB protein includes 2 [4Fe—4S] clusters per polypeptide chain. Note that the upper cluster in FIG. 3, the auxiliary cluster, has a 5^{th} coordinated sulfur atom. This additional sulfur atom is hypothesized to be donated to make the tetrahydrothiophene ring of the biotin. Biotin synthases comprising 2 [4Fe—4S] clusters per polypeptide chain are termed “Type II biotin synthases” herein, while the previously identified biotin synthases containing 1 [4Fe—4S] cluster and 1[2Fe—2S] cluster per polypeptide chain are termed “Type I biotin synthases”.

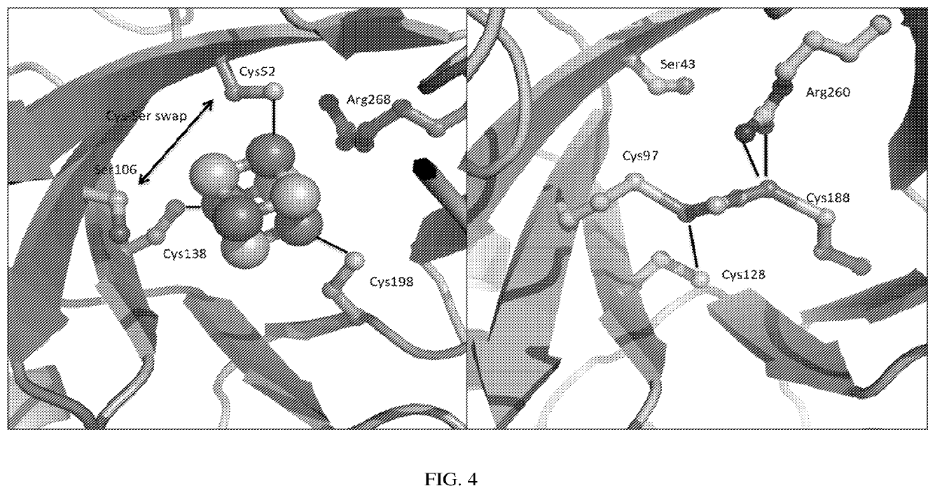

The active sites of E. coli BioB and the B. obeum BioB are compared in FIG. 4. The E. coli BioB and the B. obeum BioB active sites have significant differences. Notably, the B. obeum BioB shows a bound auxiliary [4Fe—4S] cluster coordinating with residues C-52, C-138, and C-198, while the E. coli BioB structure contains an auxiliary [2Fe—2S] cluster coordinating with residues C-97, C-128, C-188, and R-260. Overall RMSD is 1.6 Å between the E. coli BioB and the B. obeum BioB protein structures.

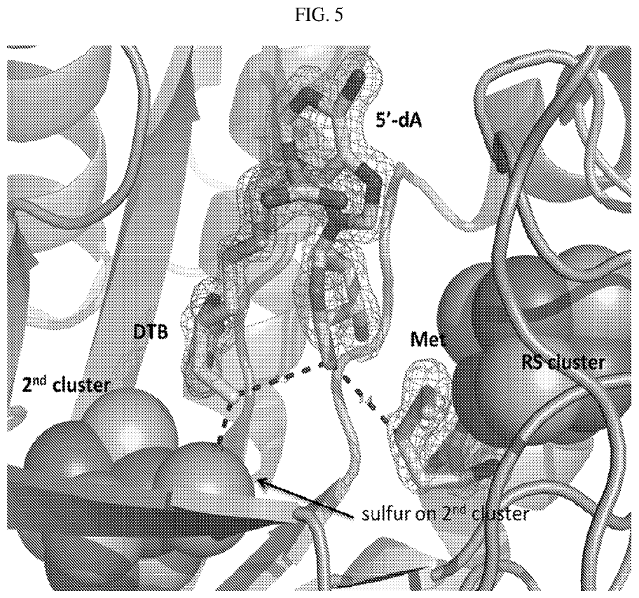

FIG. 5 shows the B. obeum BioB active site with DTB and SAM after a dithionite (Na₂O₄S₂) soak. Resolution of the data in FIG. 5 is 1.51 Å, with R=0.1724 and R_{free}=0.1999. Unexpectedly, the auxiliary cluster (2^{nd} cluster) includes a fifth sulfur atom coordinated to one of the Fe atoms and within proximity to the bound dethiobiotin.

### Example 2. In Vitro Enzyme Activity of Type I and Type II BioBs

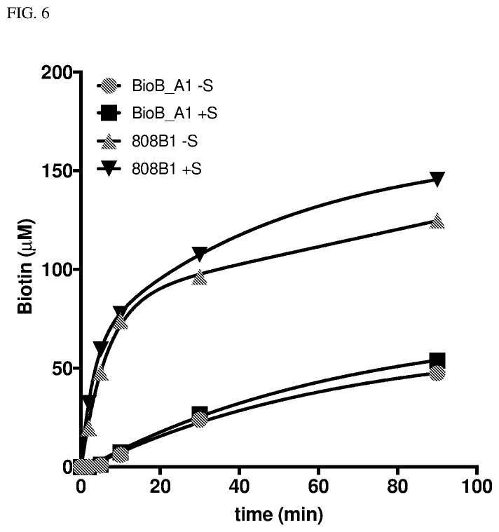

FIG. 6 is a graph showing enzymatic synthesis of biotin as a function of time by two different BioB enzymes in the presence or absence of added Sulfur, Type-II BioB from Blautia obeum (triangles) and Type-I BioB from Methylococcus capsulatus (circles/squares). Assays contained 200 μM of the biotin synthase with 1 mM SAM, 2 mM desthiobiotin, 5 μM flavodoxin reductase, and 25 μM flavodoxin. Reactions were initiated by adding NADPH to a final concentration of 1 mM. Each reaction was performed at 22° C. in the absence or presence of 1 mM free Na₂S. The B. obeum Type II BioB is about 10-fold faster than the M. capsulatus Type I BioB under these conditions. Excess sulfur did not significantly increase number of turnovers for either enzyme in 90 minutes under these conditions, however the Type II BioB did show somewhat enhanced biotin production in the presence of the added sulfur at the later time points.

### Example 3. Veillonella Parvula HSIVP1 BioB is a Type II BioB

When the sequence of the V. parvula BioB (Uniprot Accession TOTAB9; SEQ ID NO:9)) is aligned with the E. coli Type I BioB sequence (SEQ ID NO:13), the residues corresponding to E. coli BioB residues S-43 and C-97 in V. parvula BioB (C-69 and S-123) showed the occurrence of a cys-ser swap, indicating that V. parvula BioB is a Type II BioB.

This identification was confirmed by growing crystals of the V. parvula BioB, and determining the structure by X-ray crystallography, by procedures in general accordance with those described above for the B. obeum BioB. The structure of the V. parvula BioB showed the presence of two [4Fe—4S] clusters, the RS cluster and the 2^{nd} cluster, per polypeptide chain confirming the identification by sequence alignment that the V. parvula BioB is a Type II BioB.

The disclosure herein include(s) at least the following aspects:

Aspect 1. A method of producing biotin comprises contacting desthiobiotin with a Type II biotin synthase (Type II BioB) holo-protein in vitro under conditions effective to produce biotin; and recovering the biotin, wherein the Type II biotin synthase holo-protein comprises, per polypeptide chain, a first [4Fe—4S] cluster (radical SAM (RS) cluster) coordinated to a CxxxCxxC motif in the polypeptide chain and a second [4Fe—4S] cluster.

Aspect 2. The method of aspect 1, wherein the Type II biotin synthase is a Blautia sp. biotin synthase, a Clostridium sp. biotin synthase, a Bacteroides sp. biotin synthase, a Porphyromonas sp. biotin synthase, a Veillonella sp. biotin synthase, a Cyanothece sp. biotin synthase, an Akkermansia sp. biotin synthase, or a combination thereof

Aspect 3. The method of aspect 1 or 2, wherein the Type II biotin synthase is a Blautia obeum biotin synthase (SEQ ID NO:1), a Clostridium sp. HMSC19B10 biotin synthase (SEQ ID NO:2), a Bacteroides caccae (ATCC 43185) biotin synthase (SEQ ID NO:3), a [Clostridium] spiroforme DSM 1552 biotin synthase (SEQ ID NO:4), a Porphyromonas gingivalis (strain ATCC BAA-308/W83) biotin synthase (SEQ ID NO:5), a Bacteroides cellulosilyticus biotin synthase (SEQ ID NO:6), a Clostridium perfringens biotin synthase (SEQ ID NO:7), a Clostridium thermocellum biotin synthase (SEQ ID NO:8), a Veillonella parvula HSIVP1 biotin synthase (SEQ ID NO:9), a Cyanothece sp. (strain ATCC 51142) biotin synthase (SEQ ID NO:10), a Porphyromonas gingivalis (strain ATCC 33277) biotin synthase (SEQ ID NO:11), an Akkermansia muciniphila (strain ATCC BAA-835/Muc) biotin synthase (SEQ ID NO:12), or a combination thereof.

Aspect 4. The method of any one of aspects 1 to 3, wherein the conditions comprise S-adenosylmethionine (SAM), NADPH, and one or more polypeptides to mediate transfer of an electron from NADPH to the radical SAM [4Fe—4S] cluster

Aspect 5. The method of aspect 4, wherein the one or more polypeptides to mediate transfer of an electron from NADPH to the radical SAM [4Fe—4S] cluster comprises a flavodoxin/ferredoxin-NADP reductase; a pyruvate-flavodoxin/ferredoxin oxidoreductase; a flavodoxin; a ferredoxin; or a combination thereof.

Aspect 6. The method of any one of aspects 1 to 5, wherein the conditions comprise a temperature of 30 C to 45 C.

Aspect 7. The method of any one of aspects 1 to 6, wherein the conditions comprise added Na₂S

Aspect 8. The method of any one of aspects 1 to 7, wherein the contacting occurs in a cell-free system

Aspect 9. The method of any one of aspects 1 to 8, wherein the Type II biotin synthase holo-protein is a B. obeum Type II biotin synthase holo-protein.

Aspect 10. A recombinant microorganism comprises a transgene encoding a polypeptide of a Type II biotin synthase, wherein a holo-protein of the Type II biotin synthase comprises per polypeptide chain a first [4Fe—4S] cluster (radical SAM (RS) cluster) coordinated to a CxxxCxxC motif in the polypeptide chain and a second [4Fe—4S] cluster.

Aspect 11. The microorganism of aspect 10 wherein the transgene is operably linked to a constitutive promoter.

Aspect 12. The microorganism of aspect 10 or 11, wherein the microorganism is a bacterium, a yeast, or a filamentous fungus.

Aspect 13. The microorganism of aspect 12, wherein the microorganism is a bacterium, wherein the bacterium species is Escherichia, Bacillus, Brevibacterium, Burkholderia, Campylobacter, Corynebacterium, Pseudomonas, Serratia, Lactobacillus, Lactococcus, Acinetobacter, Pseudomonas, or Acetobacter.

Aspect 14. The microorganism of aspect 10 or 11, wherein the microorganism is a yeast, wherein the yeast is Saccharomyces cerevisiae, Pichia pastoris, Hansenula polymorpha, Komagataella sp., Kluyveromyces lactis, and Yarrowia lipolytica.

Aspect 15. The microorganism of aspect 10 or 11, wherein the microorganism is a filamentous fungus, wherein the filamentous fungus is a species of Aspergillus, Trichoderma, Penicillium, or Rhizopus.

Aspect 16. A method for producing biotin comprises cultivating the recombinant microorganism of any one of aspects 10 to 15 in a growth medium to produce a culture; and recovering biotin from the culture.

Aspect 17. The method of aspect 16 further comprising purifying the recovered biotin; or introducing the recombinant microorganism to the growth medium.

Aspect 18. The method of aspect 16 or 17, wherein the growth medium comprises a carbon source selected from glucose, maltose, galactose, fructose, sucrose, arabinose, xylose, raffinose, mannose, lactose, and a combination thereof.

Aspect 19. The method of any one of aspects 16 to 18, wherein cultivating is performed under conditions to produce biotin.

Aspect 20. The method of any one of aspects 16 to 19, wherein the recovered biotin is increased compared to recovered biotin from cultivating the same microorganism not expressing the Type II biotin synthase holo-protein.

Aspect 21. A method of producing biotin comprising aligning a query sequence with a reference sequence, wherein the reference sequence is a known Type I biotin synthase (Type I BioB) holo-protein, preferably an E. coli K12 Type 1 biotin synthase holo-protein sequence (SEQ ID NO: 13), which reference sequence contains Serine at amino acid 43 (Ser-43) and Cysteine at amino acid 97 (Cys-97);

identifying the amino acid of the query sequence corresponding to Ser-43 of the reference sequence to provide query corresponding amino acid 43;

identifying the amino acid of the query sequence corresponding to Cys-97 of the reference sequence to provide query corresponding amino acid 97;

classifying the query sequence as a Type II biotin synthase holo-protein if the query corresponding amino acid 43 is Cysteine and the query corresponding amino acid 97 is Serine to provide an identified Type II biotin synthase holo-protein sequence;

contacting desthiobiotin with a protein having the amino acid sequence of the identified Type II biotin synthase holo-protein sequence in vitro under conditions effective to produce biotin; and recovering the biotin.

Aspect 22. The method of any one of aspects 1-8, wherein the Type II biotin synthase holo-protein is a protein identified as having a Cysteine at the amino acid position corresponding to Ser-43 in SEQ ID NO: 13 and having a Serine at the amino acid position corresponding to Cys-97 in SEQ ID NO:13.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

Unless otherwise specified herein, any reference to standards, regulations, testing methods and the like, refer to the standard, regulation, guidance, or method that is in force at the time of filing of the present application.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of producing biotin comprising
contacting desthiobiotin with a Type II biotin synthase (Type II BioB) holo-protein in vitro under conditions effective to produce biotin; and
recovering the biotin,
wherein the Type II biotin synthase holo-protein comprises per polypeptide chain a first [4Fe—4S] cluster (radical SAM (RS) cluster) coordinated to a CxxxCxxC motif in the polypeptide chain and a second [4Fe—4S] cluster.

2. The method of claim 1, wherein the Type II biotin synthase is a Blautia sp. biotin synthase, a Clostridium sp. biotin synthase, a Bacteroides sp. biotin synthase, a Porphyromonas sp. biotin synthase, a Veillonella sp. biotin synthase, a Cyanothece sp. biotin synthase, an Akkermansia sp. biotin synthase, or a combination thereof.

3. The method of claim 1, wherein the Type II biotin synthase is a Blautia obeum biotin synthase (SEQ ID NO:1), a Clostridium sp. HMSC19B10 biotin synthase (SEQ ID NO:2), a Bacteroides caccae (ATCC 43185) biotin synthase (SEQ ID NO:3), a [Clostridium] spiroforme DSM1552 biotin synthase (SEQ ID NO:4), a Porphyromonas gingivalis (strain ATCC BAA-308 W83) biotin synthase (SEQ ID NO: 5), a Bacteroides cellulosilyticus biotin synthase (SEQ ID NO:6), a Clostridium perfringens biotin synthase (SEQ ID NO:7), a Clostridium thermocellum biotin synthase (SEQ ID NO:8), a Veillonella parvula HSIVP1 biotin synthase (SEQ ID NO:9), a Cyanothece sp. (strain ATCC 51142) biotin synthase (SEQ ID NO:10), a Porphyromonas gingivalis (strain ATCC 33277) biotin synthase (SEQ ID NO:11), an Akkermansia muciniphila (strain ATCC BAA-835 Muc) biotin synthase (SEQ ID NO:12), a Type II biotin synthase as listed in TABLE 1 of the specification, or a combination thereof.

4. The method of claim 1, wherein the conditions comprise S-adenosylmethionine (SAM), NADPH, and one or more polypeptides to mediate transfer of an electron from NADPH to the radical SAM [4Fe—4S] cluster; wherein the one or more polypeptides to mediate transfer of an electron from NADPH to the radical SAM [4Fe—4S] cluster comprises a flavodoxin/ferredoxin-NADP reductase; a pyruvate-flavodoxin/ferredoxin oxidoreductase; a flavodoxin; a ferredoxin; or a combination thereof.

5. (canceled)

6. The method of claim 1, wherein the conditions comprise a temperature of 15 C to 45 C.

7. The method of claim 1, wherein the conditions comprise added Na₂S.

8. The method of claim 1, wherein the contacting occurs in a cell-free system.

9. The method of claim 1, wherein the Type II biotin synthase holo-protein is a B. obeum Type II biotin synthase holo-protein.

10. A recombinant microorganism comprising a transgene encoding a polypeptide of a Type II biotin synthase,
wherein a holo-protein of the Type II biotin synthase comprises per polypeptide chain a first [4Fe—4S] cluster (radical SAM (RS) cluster) coordinated to a CxxxCxxC motif in the polypeptide chain and a second [4Fe—4S] cluster.

11. The microorganism of claim 10 wherein the transgene is operably linked to a constitutive promoter.

12. The microorganism of claim 10, wherein the microorganism is a bacterium, a yeast, or a filamentous fungus.

13. The microorganism of claim 12, wherein the microorganism is a bacterium, wherein the bacterium species is Escherichia, Bacillus, Brevibacterium, Burkholderia, Campylobacter, Corynebacterium, Pseudomonas, Serratia, Lactobacillus, Lactococcus, Acinetobacter, Pseudomonas, or Acetobacter.

14. The microorganism of claim 12, wherein the microorganism is a yeast, wherein the yeast is Saccharomyces cerevisiae, Pichia pastoris, Hansenula polymorpha, Komagataella sp., Kluyveromyces lactis, and Yarrowia lipolytica.

15. The microorganism of claim 12, wherein the microorganism is a filamentous fungus, wherein the filamentous fungus is a species of Aspergillus, Trichoderma, Penicillium, or Rhizopus.

16. A method for producing biotin, comprises:
cultivating the recombinant microorganism of claim 10 in a growth medium to produce a culture;
recovering biotin from the culture; and further comprising
purifying the recovered biotin; or
introducing the recombinant microorganism to the growth medium.

17. (canceled)

18. The method of claim 16, wherein the growth medium comprises a carbon source selected from glucose, maltose, galactose, fructose, sucrose, arabinose, xylose, raffinose, mannose, lactose, and a combination thereof.

19. The method of claim 16, wherein
cultivating is performed under conditions to produce biotin.

20. The method of claim 16, wherein the recovered biotin is increased compared to recovered biotin from cultivating the same microorganism not expressing the Type II biotin synthase holo-protein.

21. A method of producing biotin comprising
aligning a query sequence with a reference sequence, wherein the reference sequence is a known Type I biotin synthase (Type I BioB) holo-protein, preferably an E. coli K12 Type 1 biotin synthase holo-protein sequence (SEQ ID NO: 13), which reference sequence contains Serine at amino acid 43 (Ser-43) and Cysteine at amino acid 97 (Cys-97);
identifying the amino acid of the query sequence corresponding to Ser-43 of the reference sequence to provide query corresponding amino acid 43;
identifying the amino acid of the query sequence corresponding to Cys-97 of the reference sequence to provide query corresponding amino acid 97;
classifying the query sequence as a Type II biotin synthase holo-protein if the query corresponding amino acid 43 is Cysteine and the query corresponding amino acid 97 is Serine to provide an identified Type II biotin synthase holo-protein sequence;
contacting desthiobiotin with a protein having the amino acid sequence of the identified Type II biotin synthase holo-protein sequence in vitro under conditions effective to produce biotin; and recovering the biotin.

22. The method of claim 1, wherein the Type II biotin synthase holo-protein is a protein identified as having a Cysteine at the amino acid position corresponding to Ser-43 in SEQ ID NO: 13 and having a Serine at the amino acid position corresponding to Cys-97 in SEQ ID NO:13.
